SEQUENCE LISTING (7 sequences)

SEQ ID NO: 1 - protein, 327 aa, Erwinia chrysanthemi
ADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 2 - protein, 20 aa, Artificial Sequence
  Sythetic PA(20) peptide
AAPAAPAPAAPAAPAPAAPA

SEQ ID NO: 3 - protein, 40 aa, Artificial Sequence
  Synthetic PA(40) peptide
AAPAAPAPAAPAAPAPAAPAAAPAAPAPAAPAAPAPAAPA

SEQ ID NO: 4 - protein, 20 aa, Artificial Sequence
  Synthetic PA(20) peptide
AAPAAPAPAAPAAPAPAAPA

SEQ ID NO: 5 - protein, 40 aa, Artificial Sequence; identical to SEQ ID NO: 3
  Synthetic PA(40)-polypeptide

SEQ ID NO: 6 - protein, 528 aa, Artificial Sequence
  Synthetic  PA(200)-fusion protein
AAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 7 - protein, 728 aa, Artificial Sequence
  Synthetic PA(400) peptide
AAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

(19) United States
(12) Patent Application Publication
(10) Pub. No.: US 2023/0173042 A1
(43) Pub. Date: Jun. 8, 2023

(54) METHODS OF TREATMENT WITH ASPARAGINASE

(71) Applicant: JAZZ PHARMACEUTICALS IRELAND LTD., Dublin (IE)

(72) Inventors: Polly PINE, Palo Alto, CA (US); Hemamalini GURSAHANI, Palo Alto, CA (US)

(21) Appl. No.: 16/767,920

(22) PCT Filed: Nov. 30, 2018
(86) PCT No.: PCT/US2018/063448

§ 371 Date: May 28, 2020

Related U.S. Application Data
(60) Provisional application No. 62/592,982, filed on Nov. 30, 2017.
(60) Provisional application No. 62/625,078, filed on Feb. 1, 2018.
(60) Provisional application No. 62/631,142, filed on Feb. 15, 2018.
(60) Provisional application No. 62/673,075, filed on May 17, 2018.

(51) Int. Cl. A61K 38/50; A61K 47/68; A61K 47/60

(52) U.S. Cl. CPC A61K 38/50; A61K 47/6815; A61K 47/60; C12Y 305/01001

(57) ABSTRACT

The invention relates to methods of treating diseases with L-asparaginase.

### FIELD OF THE INVENTION

The present invention concerns a conjugate of a protein having substantial L-asparagine aminohydrolase activity and polyethylene glycol, particularly wherein the polyethylene glycol has a molecular weight less than or equal to about 5000 Da, particularly a conjugate wherein the protein is a L-asparaginase from Erwinia, and its use in therapy.

### BACKGROUND OF THE INVENTION

Proteins with L-asparagine aminohydrolase activity, commonly known as L-asparaginases, have successfully been used for the treatment of Acute Lymphoblastic Leukemia (ALL) in children for many years. ALL is the most common childhood malignancy (Avramis and Panosyan, (2005) 44:367-393).

L-asparaginase has also been used to treat Hodgkin's disease, acute myelocytic Leukemia, acute myclomonocytic Leukemia, chronic lymphocytic Leukemia, lymphosarcoma, reticulosarcoma, and melanosarcoma (Kotzia (2007) J. Biotechnol. 127, 657-669). The anti-tumor activity of L-asparaginase is believed to be due to the inability or reduced ability of certain malignant cells to synthesize L-asparagine (Kotzia (2007) J. Biotechnol. 127, 657-669). These malignant cells rely on an extracellular supply of L-asparagine. However, the L-asparaginase enzyme catalyzes the hydrolysis of L-asparagine to aspartic acid and ammonia, thereby depleting circulating pools of L-asparagine and killing tumor cells which cannot perform protein synthesis without L-asparagine (Kotzia (2007) J. Biotechnol. 127, 657-669).

L-asparaginase from E. coli was the first enzyme drug used in ALL therapy and has been marketed as Elspar® in the United States or as Kidrolase® and L-asparaginase Medac® in Europe. L-asparaginases have also been isolated from other microorganisms, e.g., an L-asparaginase protein from Erwinia chrysanthemi, named crisantaspase, that has been marketed as Erwinase® (Wriston (1985) Meth. Enzymol. 113, 608-618; Goward (1992) Bioseparation 2, 335-341). L-asparaginases from other species of Erwinia have also been identified, including, for example, Erwinia chrysanthemi 3937 (Genbank Accession No. AAS67028), Erwinia chrysanthemi NCPPB 1125 (Genbank Accession No. CAA31239), Erwinia carotovora (Genbank Accession No. AAP92666), and Erwinia carotovora subsp. astroseptica (Genbank Accession No. AAS67027). These Erwinia chrysanthemi L-asparaginases have about 91-98% amino acid sequence identity with each other, while the Erwinia carotovora L-asparaginases have approximately 75-77% amino acid sequence identity with the Erwinia chrysanthemi L-asparaginases (Kotzia (2007) J. Biotechnol. 127 657-669).

The currently available L-asparaginase preparations do not provide alternative or complementary therapies, particularly therapies to treat ALL, that are characterized by high catalytic activity and significantly improved pharmacological and pharmacokinetic properties, as well as reduced immunogenicity.

In one aspect, the problem to be solved by the invention is to provide an L-asparaginase preparation with: high in vitro bioactivity; a stable PEG-protein linkage; prolonged in vivo half-life; significantly reduced immunogenicity, as evidenced, for example, by the reduction or elimination of an antibody response against the L-asparaginase preparation following repeated administrations; and usefulness as a second-line therapy for patients who have developed sensitivity to first-line therapies using, e.g., E. coli-derived L-asparaginases.

This problem has not been solved by known L-asparaginase conjugates, which either have significant cross-reactivity with modified L-asparaginase preparations (Wang (2003) Leukemia 17, 1583-1588 incorporated herein by reference in its entirety), or which have considerably reduced in vitro activity (Kuchumova (2007) Biochemistry (Moscow) Supplement Series B: Biomedical Chemistry, 1, 230-232 incorporated herein by reference in its entirety). This problem is solved according to the present invention by providing a conjugate of Erwinia L-asparaginase with a hydrophilic polymer, more specifically, a polyethylene glycol with a molecular weight of 5000 Da or less, a method for preparing such a conjugate and the use of the conjugate.

### SUMMARY OF THE INVENTION

The invention encompasses a method of treating a disease treatable by L-asparagine depletion in a patient comprising administering an effective amount conjugate of a protein having substantial L-asparagine aminohydrolase activity and polyethylene glycol (PEG), wherein the polyethylene glycol has a molecular weight less than or equal to about 5000 Da, wherein the protein is a L-asparaginase from Erwinia. In some embodiments, the L-asparaginase has at least about 80%, 85%, 90%, 91%, 92%, 93%, 94%, 95%, 96%, 97%, 98%, 99%, or 100% sequence identity to the amino acid of SEQ ID NO: 1. In some embodiments, the conjugate comprises an L-asparaginase from Erwinia having at 100% sequence identity to the amino acid of SEQ ID NO: 1. In some embodiments, the PEG has a molecular weight of about 5000 Da, 4000, Da, 3000 Da, 2500 Da, or 2000 Da. In some embodiments, the conjugate has an in vitro activity of at least 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the L-asparaginase when not conjugated to PEG. In some embodiments, the conjugate has an L-asparagine depletion activity at least about 10, 20, 30, 40, 50, 60, 70, 80, 90, or 100 times more potent than the L-asparaginase when not conjugated to PEG. In some embodiments, the conjugate depletes plasma L-asparagine levels to an undetectable level for at least about 12, 24, 48, 96, 108, or 120 hours. In some embodiments, the conjugate has a longer in vivo circulating half-life compared to the L-asparaginase when not conjugated to PEG. In some embodiments, the conjugate has a longer t½ than pegaspargase administered at an equivalent protein dose. In some embodiments, the conjugate has a t½ of at least about 58 to about 65 hours at a dose of about 50 μg/kg on a protein content basis, and a t½ of at least about 34 to about 40 hours at a dose of about 10 μg/kg on a protein content basis, following iv administration in mice. In some embodiments, the conjugate has a t½ of at least about 100 to about 200 hours at a dose ranging from about 10,000 to about 15,000 IU/m² (about 20-30 mg protein/m²). In some embodiments, the conjugate has a greater area under the curve (AUC) compared to the L-asparaginase when not conjugated to PEG. In some embodiments, the conjugate has a mean AUC that is at least about 3 times greater than pegaspargase at an equivalent protein dose. In some embodiments, the PEG is covalently linked to one or more amino groups of the L-asparaginase. In some embodiments, the PEG is covalently linked to the one or more amino groups by an amide bond. In some embodiments, the PEG is covalently linked to at least from about 40% to about 100% of the accessible amino groups or at least from about 40% to about 90% of total amino groups.

The method of the invention encompass use of conjugate having the formula:

Asp-[NH—CO—(CH₂)ₓ—CO—NH-PEG]ₙ

wherein Asp is the L-asparaginase, NH is one or more of the NH groups of the lysine residues and/or the N-terminus of the Asp, PEG is a polyethylene glycol moiety, n is a number that represents at least about 40% to about 100% of the accessible amino groups in the Asp, and x is an integer ranging from about 1 to about 8, more specifically, from about 2 to about 5. In a specific embodiment, the PEG is monomethoxy-polyethylene glycol (mPEG).

The method of the invention encompass use of a conjugate of L-asparaginase which comprises one or more peptide(s), wherein each is independently a peptide R^{N}-(P/A)-R^{C}, wherein (P/A) is an amino acid sequence consisting solely of proline and alanine amino acid residues, wherein R^{N} is a protecting group attached to the N-terminal amino group of the amino acid sequence, and wherein R^{C} is an amino acid residue bound via its amino group to the C-terminal carboxy group of the amino acid sequence, wherein each peptide is conjugated to the L-asparaginase via an amide linkage formed from the carboxy group of the C-terminal amino acid residue R^{C} of the peptide and a free amino group of the L-asparaginase, and wherein at least one of the free amino groups, which the peptides are conjugated to, is not an N-terminal α-amino group of the L-asparaginase.

The method of the invention encompass use of the conjugate for the treatment of cancer. In some embodiments, the cancer is selected from the group consisting of lymphoma, large cell immunoblastic lymphoma, non-Hodgkin's lymphoma, diffuse large B-cell lymphoma, NK lymphoma, Hodgkin's disease, acute myelocytic Leukemia, acute promyelocytic Leukemia, acute myelomonocytic Leukemia, acute monocytic Leukemia, acute T-cell Leukemia, acute myeloid Leukemia (AML), biphenotypic B-cell myelomonocytic Leukemia and chronic lymphocytic Leukemia.

In some embodiments, the disease is selected from the group consisting of renal cell carcinoma, renal cell adenocarcinoma, glioblastoma including glioblastoma multiforma and glioblastoma astrocytoma, medulloblastoma, rhabdomyosarcoma, malignant melanoma, epidermoid carcinoma, squamous cell carcinoma, lung carcinoma including large cell lung carcinoma and small cell lung carcinoma, endometrial carcinoma, ovarian adenocarcinoma, ovarian tetratocarcinoma, cervical adenocarcinoma, breast carcinoma, breast adenocarcinoma, breast ductal carcinoma, pancreatic adenocarcinoma, pancreatic ductal carcinoma, colon carcinoma, colon adenocarcinoma, colorectal adenocarcinoma, bladder transitional cell carcinoma, bladder papilloma, prostate carcinoma, osteosarcoma, epitheloid carcinoma of the bone, prostate carcinoma, and thyroid cancer. In some embodiments, the conjugate is administered at an amount of about 5 U/kg body weight to about 50 U/kg body weight.

In some embodiments, the conjugate is administered at a dose ranging from about 100 to about 15,000 IU/m². In some embodiments, the administration is intravenous or intramuscular and is once per week, twice per week, or three times per week. In some embodiments, conjugate is administered as monotherapy. In some embodiments, the conjugate is administered as part of a combination therapy. In some embodiments, the conjugate is administered as part of a combination therapy with Oncaspar®, daunorubicin, cytarabine, Vyxeos®, ABT-737, Venetoclax, dactolisib, bortezomib, carfilzomib, vincristine, prednisolone, everolimus, and/or CB-839. In some embodiments, the patient receiving treatment has had a previous hypersensitivity to an E. coli asparaginase or PEGylated form thereof or to an Erwinia asparaginase. In some embodiments, the patient receiving treatment has had a disease relapse, in particular a relapse that occurs after treatment with an E. coli asparaginase or PEGylated form thereof.

### BRIEF DESCRIPTION OF DRAWINGS

FIG. 1 and FIG. 2 depict in vivo experimental data using pegcrisantaspase with other compounds.

FIG. 3 depicts dose-response curves with exemplary single agents.

FIG. 4 depicts dose-response curves with exemplary mixtures with inactive agents

FIG. 5 depicts comparison data for the exemplary single agents and mixtures.

FIG. 6 depicts a dose-oriented plot indicating whether drug combinations are synergistic.

FIG. 7 depicts CNS cell line data.

FIG. 8 and FIG. 9 depict IC₅₀ effect of pegcrisantaspase.

FIG. 10 depicts in vitro sensitivity of pegcrisantaspase in leukemia and lymphoma cell lines.

FIG. 11A-FIG. 11C depicts mPEG-r-crisantaspase conjugate (Pegcrisantaspase) tested against various cell lines.

FIG. 12 depicts the results of a T-test.

FIG. 13A-FIG. 13B depicts mPEG-r-crisantaspase conjugate (Pegcrisantaspase and Oncaspar®) tested with other agents used in the standard of care for AML or DLBCL.

FIG. 14A-FIG. 14B depicts Pegcrisantaspase combined with reference compounds in AML and DLBCL cell lines.

FIG. 15 depicts testing of Erwinase in Oncolines™ and testing of Oncaspar in Oncolines™.

FIG. 16 depicts testing of PEG-crisantaspace Oncolines™.

FIG. 17 depicts testing of PA-20 Corynebacterium in Oncolines™ and testing of PA-20 Corynebacterium in Oncolines™.

FIG. 18 depicts testing of PA-20 Psuedomonas in Oncolines™.

FIG. 19 depicts testing of PA-40 Psuedomonas in Oncolines™ and PA-200 Psuedomonas in Oncolines™.

### DETAILED DESCRIPTION OF THE INVENTION

L-asparaginases of bacterial origin have a high immunogenic and antigenic potential and frequently provoke adverse reactions ranging from mild allergic reaction to anaphylactic shock in sensitized patients (Wang (2003) Leukemia 17, 1583-1588). E. coli L-asparaginase is particularly immunogenic, with reports of the presence of anti-asparaginase antibodies to E. coli L-asparaginase following i.v. or i.m. administration reaching as high as 78% in adults and 70% in children (Wang (2003) Leukemia 17, 1583-1588).

L-asparaginases from Escherichia coli and Erwinia chrysanthemi differ in their pharmacokinetic properties and have distinct immunogenic profiles, respectively (Klug Albertsen (2001) Brit. J. Haematol. 115, 983-990). Furthermore, it has been shown that antibodies that developed after a treatment with L-asparaginase from E. coli do not cross react with L-Asparaginase from Erwinia (Wang (2003) Leukemia 17, 1583-1588). Thus, L-asparaginase from Erwinia crisantaspase has been used as a second line treatment of ALL in patients that react to E. coli L-asparaginase (Duval (2002) Blood 15, 2734-2739; Avramis (2005) Clin. Pharmacokinet. 44, 367-393).

In another attempt to reduce immunogenicity associated with administration of microbial L-asparaginases, an E. coli L-asparaginase has been developed that is modified with methoxy-polyethyleneglycol (mPEG). This method is commonly known as “PEGylation” and has been shown to alter the immunological properties of proteins (Abuchowski (1977) J. Biol. Chem. 252, 3578-3581). This so-called mPEG-L-asparaginase, or pegaspargase, marketed as Oncaspar® was first approved in the U.S. for second line treatment of ALL in 1994, and has been approved for first-line therapy of ALL in children and adults since 2006. Oncaspar® has a prolonged in vivo half-life and a reduced immunogenicity/antigenicity.

Oncaspar® is E. coli L-asparaginase that has been modified at multiple lysine residues using 5 kDa mPEG-succinimidyl succinate (SS-PEG) (U.S. Pat. No. 4,179,337). SS-PEG is a PEG reagent of the first generation that contains an instable ester linkage that is sensitive to hydrolysis by enzymes or at slightly alkaline pH values (U.S. Pat. No. 4,670,417). These properties decrease both in vitro and in vivo stability and can impair drug safety.

Furthermore, it has been demonstrated that antibodies developed against L-asparaginase from E. coli will cross react with Oncaspar® (Wang (2003) Leukemia 17, 1583-1588). Even though these antibodies were not neutralizing, this finding clearly demonstrated the high potential for cross-hypersensitivity or cross-inactivation in vivo. Indeed, in one report 30-41% of children who received pegaspargase had an allergic reaction (Wang (2003) Leukemia 17, 1583-1588).

In addition to outward allergic reactions, the problem of “silent hypersensitivity” was recently reported, whereby patients develop anti-asparaginase antibodies without showing any clinical evidence of a hypersensitivity reaction (Wang (2003) Leukemia 17, 1583-1588). This reaction can result in the formation of neutralizing antibodies to E. coli L-asparaginase and pegaspargase; however, these patients are not switched to Erwinia L-asparaginase because there are not outward signs of hypersensitivity, and therefore they receive a shorter duration of effective treatment (Holcenberg (2004) Pediatr. Hematol. Oncol. 26, 273-274).

Erwinia chrysanthemi L-asparaginase treatment is often used in the event of hypersensitivity to E. coli-derived L-asparaginases. However, it has been observed that as many as 30-50% of patients receiving Erwinia L-asparaginase arc antibody-positive (Avramis (2005) Clin. Pharmacokinet. 44, 367-393). Moreover, because Erwinia chrysanthemi L-asparaginase has a significantly shorter elimination half-life than the E. coli L-asparaginases, it must be administered more frequently (Avramis (2005) Clin. Pharmacokinet. 44, 367-393). In a study by Avramis, Erwinia asparaginase was associated with inferior pharmacokinetic profiles (Avramis (2007) J. Pediatr. Hematol. Oncol. 29, 239-247). E. coli L-asparaginase and pegaspargase therefore have been the preferred first-line therapies for ALL over Erwinia L-asparaginase.

Numerous biopharmaceuticals have successfully been PEGylated and marketed for many years. In order to couple PEG to a protein, the PEG has to be activated at its OH terminus. The activation group is chosen based on the available reactive group on the protein that will be PEGylated. In the case of proteins, the most important amino acids are lysine, cysteine, glutamic acid, aspartic acid, C-terminal carboxylic acid and the N-terminal amino group. In view of the wide range of reactive groups in a protein nearly the entire peptide chemistry has been applied to activate the PEG moiety. Examples for this activated PEG-reagents are activated carbonates, e.g., p-nitrophenyl carbonate, succinimidyl carbonate; active esters, e.g., succinimidyl ester; and for site specific coupling aldehydes and maleimides have been developed (Harris (2002) Adv. Drug Del. Rev. 54, 459-476). The availability of various chemical methods for PEG modification shows that each new development of a PEGylated protein will be a case by case study. In addition to the chemistry the molecular weight of the PEG that is attached to the protein has a strong impact on the pharmaceutical properties of the PEGylated protein. In most cases it is expected that, the higher the molecular weight of the PEG, the better the improvement of the pharmaceutical properties (Sherman (2008) Adv. Drug Del. Rev. 60, 59-68; Holtsberg (2002) Journal of Controlled Release 80, 259-271). For example, Holtsberg et al. found that, when PEG was conjugated to arginine deaminase, another amino acid degrading enzyme isolated from a microbial source, pharmacokinetic and pharmacodynamic function of the enzyme increased as the size of the PEG attachment increased from a molecular weight of 5000 Da to 20,000 Da (Holtsberg (2002) Journal of Controlled Release 80, 259-271).

However, in many cases, PEGylated biopharmaceuticals show significantly reduced activity compared to the unmodified biopharmaceutical (Fishburn (2008) J. Pharm. Sci., 1-17). In the case of L-asparaginase from Erwinia carotovora, it has been observed that PEGylation reduced its in vitro activity to approximately 57% (Kuchumova (2007) Biochemistry (Moscow) Supplement Series B: Biomedical Chemistry, 1, 230-232). The L-asparaginase from Erwinia carotovora has only about 75% homology to the Erwinia chrysanthemi L-asparaginase (crisantaspase). For Oncaspar® it is also known that its in vitro activity is approximately 50% compared to the unmodified E. coli L-asparaginase.

Described herein is a PEGylated L-asparaginase from Erwinia with improved pharmacological properties as compared with the unmodified L-asparaginase protein, as well as compared to the pegaspargase preparation from E. coli. The PEGylated L-asparaginase conjugate described herein, e.g., Erwinia chrysanthemi L-asparaginase PEGylated with 5000 Da molecular weight PEG, serves as a therapeutic agent particularly for use in patients who show hypersensitivity (e.g., an allergic reaction or silent hypersensitivity) to treatment with L-asparaginase or PEGylated L-asparaginase from E. coli. or unmodified L-asparaginase from Erwinia. The PEGylated L-asparaginase conjugate described herein is also useful as a therapeutic agent for use in patients who have had a disease relapse, e.g., a relapse of ALL, and have been previously treated with another form of asparaginase, e.g., with L-asparaginase or PEGylated L-asparaginase from E. coli.

As described in detail herein, the conjugate of the invention shows unexpectedly superior properties compared to known L-asparaginase preparations such as pegaspargase. For example, unmodified L-asparaginase from Erwinia chrysanthemi (crisantaspase) has a significantly lower half-life than unmodified L-asparaginase from E. coli (Avramis (2005) Clin. Pharmacokinet. 44, 367-393 incorporated herein by reference in its entirety). The PEGylated conjugate of the invention has a half-life that is greater than PEGylated L-asparaginase from E. coli at an equivalent protein dose.

### Definitions

Unless otherwise expressly defined, the terms used herein will be understood according to their ordinary meaning in the art.

As used herein, the term “including” means “including, without limitation,” and terms used in the singular shall include the plural, and vice versa, unless the context dictates otherwise.

As used herein, the term “disease treatable by depletion of asparagine” refers to a condition or disorder wherein the cells involved in or responsible for the condition or disorder either lack or have a reduced ability to synthesize L-asparagine. Depletion or deprivation of L-asparagine can be partial or substantially complete (e.g., to levels that are undetectable using methods and apparatus that arc known in the art).

As used herein, the term “therapeutically effective amount” refers to the amount of a protein (e.g., asparaginase or conjugate thereof), required to produce a desired therapeutic effect.

As used herein, the term “sequence identity” is used interchangeably with “homology” and as such can have the same meaning where appropriate.

The terms “co-administration,” “co-administering,” “administered in combination with,” “administering in combination with,” “simultaneous,” and “concurrent,” as used herein, encompass administration of two or more active pharmaceutical ingredients to a human subject so that both active pharmaceutical ingredients and/or their metabolites are present in the human subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present is also encompassed in the methods of the invention.

### L-Asparaginase Protein

The protein according to the invention is an enzyme with L-asparagine aminohydrolase activity, namely an L-asparaginase.

Many L-asparaginase proteins have been identified in the art, isolated by known methods from microorganisms. (See, e.g., Savitri (2003) Indian J. Biotechnol 2, 184-194 incorporated herein by reference in its entirety). The most widely used and commercially available L-asparaginases are derived from E. coli or from Erwinia chrysanthemi, both of which share 50% or less structural homology. Within the Erwinia species, typically 75-77% sequence identity was reported between Erwinia chrysanthemi and Erwinia carotovora-derived enzymes, and approximately 90% sequence identity was found between different subspecies of Erwinia chrysanthemi (Kotzia G A, Labrou E, Journal of Biotechnology (2007) 127:657-669, incorporated herein by reference in its entirety). Some representative Erwinia L-asparaginases include, for example, those provided in Table 1:

TABLE 1

| Species | Accession No. | % Identity |
|---|---|---|
| Erwinia chrysanthemi 3937 | AAS67028 | 91% |
| Erwinia chrysanthemi NCPPB 1125 | CAA31239 | 98% |
| Erwinia carotovora subsp. astroscptica | AAS67027 | 75% |
| Erwinia carotovora | AAP92666 | 77% |

The sequences of the Erwinia L-asparaginases and the GenBank entries of Table 1 are herein incorporated by reference. Preferred L-asparaginases used in therapy are L-asparaginase isolated from E. coli and from Erwinia, specifically, Erwinia chrysanthemi.

The L-asparaginases may be native enzymes isolated from the microorganisms. They can also be produced by recombinant enzyme technologies in producing microorganisms such as E. coli. As examples, the protein used in the conjugate of the invention can be a protein form E. coli produced in a recombinant E. coli producing strain, of a protein from an Erwinia species, particularly Erwinia chrysanthemi, produced in a recombinant E. coli producing strain.

Enzymes can be identified by their specific activities. This definition thus includes all polypeptides that have the defined specific activity also present in other organisms, more particularly in other microorganisms. Often enzymes with similar activities can be identified by their grouping to certain families defined as PFAM or COG. PFAM (protein family database of alignments and hidden Markov models; pfam.sanfferac.ukl) represents a large collection of protein sequence alignments. Each PFAM makes it possible to visualize multiple alignments, see protein domains, evaluate distribution among organisms, gain access to other databases, and visualize known protein structures. COGs (Clusters of Orthologous Groups of proteins; vv-ww.nebi.nlm.nih.gov/COG/) are obtained by comparing protein sequences from 43 fully sequenced genomes representing 30 major phylogenetic lines. Each COG is defined from at least three lines, which permits the identification of former conserved domains.

The means of identifying homologous sequences and their percentage homology or sequence identity are well known to those skilled in the art, and include in particular the BLAST programs, which can be used from the website blast.ncbi.olo.nih.gov/Blast.cgi with the default parameters indicated on that website. The sequences obtained can then be exploited (e.g., aligned) using, for example, the programs CLUSTALW (www.ebi.ac.uk/Tools/clustalw2/index.html) or MULTALIN (bioinfo.genotoul.fr/multalin/multalin.html) with the default parameters indicated on those websites. Using the references given on GenBank for known genes, those skilled in the art are able to determine the equivalent genes in other organisms, bacterial strains, yeasts, fungi, mammals, plants, etc. This routine work is advantageously done using consensus sequences that can be determined by carrying out sequence alignments with genes derived from other microorganisms, and designing degenerate probes to clone the corresponding gene in another organism. These routine methods of molecular biology are well known to those skilled in the art, and are described, for example, in Sambrook (2012) Molecular Cloning: A Laboratory Manual, 4th ed. Cold Spring Harbor Lab Press).

Indeed, a person skilled in the art will understand how to select and design homologous proteins retaining substantially their L-asparaginase activity. Typically, a Nessler assay is used for the determination of L-asparaginase activity according to a method described by Mashburn and Wriston (Mashburn (1963) Biochem. Biophys. Res. Comm. 12, 50 incorporated herein by reference in its entirety).

In a particular embodiment of the conjugate of the invention, the L-asparaginase protein has at least about 80% homology or sequence identity with the protein comprising the sequence of SEQ ID NO: 1, more specifically at least about 85%, 86%, 87%, 88%, 89%, 90%, 91%, 92%, 93%, 94%, 20 95%, 96%, 97%, 98%, 99%, or 100% homology or identity with the protein comprising the sequence of SEQ ID NO: 1. SEQ ID NO: 1 is as follows:

ADKLPNIVILATGGTIAGSAATGTQTTGYKAGALGV
DTLINAVPEVKKLANVKGEQFSNMASENMTGDVVL
KLSQRVNELLARDDVDGVVITHGTDTVEESAYFLH
LTVKSDKPVVFVAAMRPATAISADGPMNLLEAVRV
AGDKQSRGRGVMWLNDRIGSARYITKTNASTLDTF
KANEEGYLGVIIGNRIYYQNRIDKLHTTRSVFDVR
GLTSLPKVDILYGYQDDPEYLYDAAIQHGVKGIVY
AGIVIGAGSVSVRGIAGMRKAMEKGVVVIRSTRTG
NGIVPPDEELPGLVSDSLNPAHARILLMLALTRTS
DPKVIQEYFHTY

The term “comprising the sequence of SEQ ID NO: 1” means that the amino-acid sequence of the protein may not be strictly limited to SEQ ID NO: 1 but may contain additional amino-acids.

In a particular embodiment, the protein is the L-asparaginase of Erwinia chrysanthemi having the sequence of SEQ ID NO: 1. In another embodiment, the L-asparaginase is from Erwinia chrysanthemi NCPPB 1066 (Genbank Accession No. CAA32884 incorporated herein by reference in its entirety), either with or without signal peptides and/or leader sequences.

Fragments of the protein of SEQ ID NO: 1 are also comprised within the definition of the protein used in the conjugate of the invention. The term “a fragment of SEQ ID NO: 1” means that the sequence of the polypeptide may include less amino-acid than SEQ ID NO: 1 but still enough amino-acids to confer L-aminohydrolase activity.

It is well known in the art that a polypeptide can be modified by substitution, insertion, deletion and/or addition of one or more amino-acids while retaining its enzymatic activity. For example, substitution of one amino-acid at a given position by a chemically equivalent amino-acid that does not affect the functional properties of a protein is common. Substitutions may be defined as exchanges within one of the following groups:

Small aliphatic, non-polar or slightly polar residues: Ala, Ser, Thr, Pro, Gly;

Polar, negatively charged residues and their amides: Asp, Asn, Glu, Gln;

Polar, positively charged residues: His, Arg, Lys;

Large aliphatic, non-polar residues: Met, Leu, Ile, Val, Cys;

Large aromatic residues: Phe, Tyr, Trp.

Thus, changes that result in the substitution of one negatively charged residue for another (such as glutamic acid for aspartic acid) or one positively charged residue for another (such as lysine for arginine) can be expected to produce a functionally equivalent product.

The positions where the amino-acids are modified and the number of amino-acids subject to modification in the amino-acid sequence are not particularly limited. The skilled artisan is able to recognize the modifications that can be introduced without affecting the activity of the protein. For example, modifications in the N- or C-terminal portion of a protein may be expected not to alter the activity of a protein under certain circumstances. With respect to asparaginases, in particular, much characterization has been done, particularly with respect to the sequences, structures, and the residues forming the active catalytic site. This provides guidance with respect to residues that can be modified without affecting the activity of the enzyme. All known L-asparaginases from bacterial sources have common structural features. All are homotetramers with four active sites between the N- and C-terminal domains of two adjacent monomers (Aghaipour (2001) Biochemistry 40, 5655-5664 incorporated herein by reference in its entirety). All have a high degree of similarity in their tertiary and quaternary structures (Papageorgiou (2008) FEBS J. 275, 4306-4316 incorporated herein by reference in its entirety). The sequences of the catalytic sites of L-asparaginases are highly conserved between Erwinia chrysanthemi, Erwinia carotovora, and E. coli L-asparaginase II (Papageorgiou (2008) FEBS J. 275, 4306-4316). The active site flexible loop contains amino acid residues 14-33, and structural analysis show that Thr¹⁵, Thr⁹⁵, Ser⁶², Glu⁶³, Asp⁹⁶, and Ala¹²⁰ contact the ligand (Papageorgiou (2008) FEBS J. 275, 4306-4316). Aghaipour et al. have conducted a detailed analysis of the four active sites of Erwinia chrysanthemi L-asparaginase by examining high resolution crystal structures of the enzyme complexed with its substrates (Aghaipour (2001) Biochemistry 40, 5655-5664). Kotzia et. al provide sequences for L-asparaginases from several species and subspecies of Erwinia and, even though the proteins have only about 75-77% identity between Erwinia chrysanthemi and Erwinia carotovora, they each still have L-asparaginase activity (Kotzia (2007) J. Biotechnol. 127, 657-669 incorporated herein by reference in its entirety). Moola et. al performed epitope mapping studies of Erwinia chrysanthemi 3937 L-asparaginase and were able to retain enzyme activity even after mutating various antigenic sequences in an attempt to reduce immunogenicity of the asparaginase (Moola (1994) Biochem. J. 302, 921-927 incorporated herein by reference in its entirety). Each of the above-cited articles is herein incorporated by reference in its entirety. In view of the extensive characterization that has been performed on L-asparaginases, one of skill in the art could determine how to make fragments and/or sequence substitutions while still retaining enzyme activity.

### Polymers for Use in the Conjugate

Polymers are selected from the group of non-toxic water soluble polymers such as polysaccharides, e.g. hydroxyethyl starch, poly amino acids, e.g. poly lysine, polyester, e.g., polylactic acid, and poly alkylene oxides, e.g., polyethylene glycol (PEG).

Polyethylene glycol (PEG) or mono-methoxy-polyethyleneglycol (mPEG) is well known in the art and comprises linear and branched polymers. Examples of some polymers, particularly PEG, are provided in the following, each of which is herein incorporated by reference in its entirety: U.S. Pat. Nos. 5,672,662; 4,179,337; 5,252,714; U.S. Patent Application Publication No. 2003/0114647; U.S. Pat. Nos. 6,113,906; 7,419,600; 9,920,311 and PCT Publication No. WO2004/083258.

The quality of such polymers is characterized by the polydispersity index (PDI). The PDI reflects the distribution of molecular weights in a given polymer sample and is calculated from the weight average molecular weight divided by the number average molecular weight. It indicates the distribution of individual molecular weights in a batch of polymers. The PDI has a value always greater than 1, but as the polymer chains approach the ideal Gauss distribution (=monodispersity), the PDI approaches 1.

The polyethylene glycol has advantageously a molecular weight comprised within the range of about 500 Da to about 9,000 Da. More specifically, the polyethylene glycol (e.g, mPEG) has a molecular weight selected from the group consisting of polyethylene glycols of 2000 Da, 2500 Da, 3000 Da, 3500 Da, 4000 Da, 4500 Da, and 5000 Da. In a particular embodiment, the polyethylene glycol (e.g., mPEG) has a molecular weight of 5000 Da.

### Method for Preparing the Conjugate

For subsequent coupling of the polymer to proteins with L-asparagine aminohydrolase activity, the polymer moiety contains an activated functionality that preferably reacts with amino groups in the protein. In one aspect, the invention is directed to a method of making a conjugate, the method comprising combining an amount of polyethylene glycol (PEG) with an amount of L-asparaginase in a buffered solution for a time period sufficient to covalently link the PEG to the L-asparaginase. In a particular embodiment, the L-asparaginase is from Erwinia species, more specifically Erwinia chrysanthemi, and more specifically, the L-asparaginase comprising the sequence of SEQ ID NO: 1. In one embodiment, the PEG is monomethoxy-polyethylene glycol (mPEG).

In one embodiment, the reaction between the polyethylene glycol and L-asparaginase is performed in a buffered solution. In some particular embodiments, the pH value of the buffer solution ranges between about 7.0 and about 9.0. The most preferred pH value ranges between about 7.5 and about 8.5, e.g., a pH value of about 7.5, 7.6, 7.7, 7.8, 7.9, 8.0, 8.1, 8.2, 8.3, 8.4, or 15 8.5. In a particular embodiment, the L-asparaginase is from Erwinia species, more specifically Erwinia chrysanthemi, and more specifically, the L-asparaginase comprising the sequence of SEQ ID NO: 1.

Furthermore, PEGylation of L-asparaginase is performed at protein concentrations between about 0.5 and about 25 mg/mL, more specifically between about 2 and about 20 mg/mL and most specifically between about 3 and about 15 mg/mL. For example, the protein concentration is about 0.5, 1, 2, 3, 4, 5, 6, 7, 8, 9, 10, 11, 12, 13, 14, 15, 16, 17, 18, 19, or 20 mg/mL. In a particular embodiment, the PEGylation of L-asparaginase at these protein concentrations is of Erwinia species, more specifically Erwinia chrysanthemi, and more specifically, the L-asparaginase comprising the sequence of SEQ ID NO: 1.

At elevated protein concentrations of more than 2 mg/mL the PEGylation reaction proceeds rapidly, within less than 2 hours. Furthermore, a molar excess of polymer over amino groups in L-asparaginase of less than about 20:1 is applied. For example, the molar excess is less than about 20:1, 19:1, 18:1, 17:1, 16:1, 15:1, 14:1, 13:1, 12:1, 11:1, 10:1, 9:1, 8:1, 7.5:1, 7:1, 6.5:1, 6:1, 5.5:1, 5:1, 4.5:1, 4:1, 3.5:1, 3:1, 2.5:1, 2:1, 1.5:1, or 1:1. In a specific embodiment the molar excess is less than about 10:1 and in a more specific embodiment, the molar excess is less than about 8:1. In a particular embodiment, the L-asparaginase is from Erwinia species, more specifically Erwinia chrysanthemi, and more specifically, the L-asparaginase comprising the sequence of SEQ ID NO: 1.

The number of PEG moieties which can be coupled to the protein will be subject to the number of free amino groups and, even more so, to which amino groups are accessible for a PEGylation reaction. In a particular embodiment, the degree of PEGylation (i.e., the number of PEG moieties coupled to amino groups on the L-asparaginase) is within a range from about 10% to about 100% of free and/or accessible amino groups (e.g., about 10%, 15%, 20%, 25%, 30%, 40%, 50%, 60%, 70%, 80%, 90%, or 100%). 100% PEGylation of accessible amino groups (e.g., lysine residues and/or the N-terminus of the protein) is also referred to herein as “maximally PEGylated.” One method to determine the modified amino groups in mPEG-r-crisantaspase conjugates (degree of PEGylation) is a method described by Habeeb (A. F. S. A. Habeeb, “Determination of free amino groups in proteins by trinitrobenzensulfonic acid”, Anal. Biochem. 14 (1966), p. 328, incorporated herein by reference in its entirety). In one embodiment, the PEG moieties are coupled to one or more amino groups (wherein amino groups include lysine residues and/or the N-terminus) of the L-asparaginase. In a particular embodiment, the degree of PEGylation is within a range of from about 10% to about 100% of total or accessible amino groups (e.g., lysine residues and/or the N-terminus), e.g., about 10%, 15%, 20%, 25%, 30%, 35%, 40%, 45%, 50%, 55%, 60%, 65%, 70%, 75%, 80%, 85%, 90%, 95% or 100%. In a specific embodiment, about 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% of the total amino groups (e.g., lysine residues and/or the N-terminus) are coupled to a PEG moiety. In another specific embodiment, about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 70%, 71%, 72%, 7%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the accessible amino groups (e.g., lysine residues and/or the N-terminus) are coupled to a PEG moiety. In a specific embodiment, 40-55% or 100% of the accessible amino groups (e.g., lysine residues and/or the N-terminus) are coupled to a PEG moiety. In some embodiments, the PEG moieties are coupled to the L-asparaginase by a covalent linkage. In a particular embodiment, the L-asparaginase is from Erwinia species, more specifically Erwinia chrysanthemi, and more specifically, the L-asparaginase comprising the sequence of SEQ ID NO: 1.

In one embodiment, the conjugate of the invention can be represented by the formula

Asp-[NH—CO—(CH₂)ₓ—CO—NH-PEG]ₙ

wherein Asp is a L-asparaginase protein, NH is the NH group of a lysine residue and/or the N-terminus of the protein chain, PEG is a polyethylene glycol moiety and n is a number of at least 40% to about 100% of the accessible amino groups (e.g., lysine residues and/or the N-terminus) in the protein, all being defined above and below in the examples, x is an integer ranging from 1 to 8 (e.g., 1, 2, 3, 4, 5, 6, 7, 8), preferably 2 to 5 (e.g., 2, 3, 4, 5). In a particular embodiment, the L-asparaginase is from Erwinia species, more specifically Erwinia chrysanthemi, and more specifically, the L-asparaginase comprising the sequence of SEQ ID NO: 1.

Other methods of PEGylation that can be used to form the conjugates of the invention are provided, for example, in U.S. Pat. Nos. 4,179,337, 5,766,897, U.S. Patent Application Publication No. 2002/0065397A1, and U.S. Patent Application Publication No. 2009/0054590A1 each of which is herein incorporated by reference in its entirety.

Specific embodiments include proteins having substantial L-Asparagine aminohydrolase activity and polyethylene glycol, selected from the group of conjugates wherein:

(A) the protein has at least 90% homology of structure with the L-asparaginase from Erwinia chrysanthemi as disclosed in SEQ ID NO: 1, the polyethylene glycol has a molecular weight of about 5000 Da, the protein and polyethylene glycol moieties are covalently linked to the protein by amide bonds, and about 100% of the accessible amino groups (e.g., lysine residues and/or the N-terminus) or about 80-90%, in particular, about 84%, of total amino groups (e.g., lysine residues and/or the N-terminus) are linked to a polyethylene glycol moiety.

(B) the protein has at least 90% homology with the L-asparaginase from Erwinia chrysanthemi as disclosed in SEQ ID NO: 1, the polyethylene glycol has a molecular weight of about 5000 Da, the protein and polyethylene glycol moieties are covalently linked to the protein by amide bonds, and about 40% to about 45%, and in particular about 43% of the accessible amino groups (e.g., lysine residues and/or the N-terminus), or about 36% of the total amino groups (e.g., lysine residues and/or the N-terminus) arc linked to a polyethylene glycol moiety.

(C) the protein has at least 90% homology with the L-asparaginase from Erwinia chrysanthemi as disclosed in SEQ ID NO: 1, the polyethylene glycol has a molecular weight of about 2000 Da, the protein and polyethylene glycol moieties are covalently linked to the protein by amide bonds, and about 100% of the accessible amino groups (e.g., one or more lysine residues and/or the N-terminus) or about 80-90%, in particular, about 84% of total amino groups (e.g., lysine residues and/or the N-terminus) are linked to a polyethylene glycol moiety.

(D) the protein has at least 90% homology with the L-asparaginase from Erwinia chrysanthemi as disclosed in SEQ ID NO: 1, the polyethylene glycol has a molecular weight of about 2000 Da, the protein and polyethylene glycol moieties are covalently linked to the protein by amide bonds, and [0092] about 50% to about 60%, and in particular about 55% of the accessible amino groups (e.g., lysine residues and/or the N-terminus) or about 47% of the total amino groups (e.g., lysine residues and/or the N-terminus) are linked to a polyethylene glycol moiety.

### L-Asparaginase-PEG Conjugates

Conjugates of the invention have certain advantageous and unexpected properties compared to unmodified L-asparaginases, particularly compared to unmodified Erwinia L-asparaginases, more particularly compared to unmodified L-asparaginase from Erwinia chrysanthemi, and more particularly compared to unmodified L-asparaginase having the sequence of SEQ ID NO: 1.

In some embodiments, the methods of the invention encompass a conjugate which reduces plasma L-asparagine and glutamine levels for a time period of at least about 12, 24, 48, 72, 96, or 120 hours when administered at a dose of 5 U/kg body weight (bw) or 10 μg/kg (protein content basis). In other embodiments, the conjugate of the invention reduces plasma L-asparagine levels to undetectable levels for a time period of at least about 12, 24, 48, 72, 96, 120, or 144 hours when administered at a dose of 25 U/kg bw or 50 μg/kg (protein content basis). In other embodiments, the conjugate of the invention reduces plasma L-asparagine levels for a time period of at least about 12, 24, 48, 72, 96, 120, 144, 168, 192, 216, or 240 hours when administered at a dose of 50 U/kg bw or 100 μg/kg (protein content basis). In another embodiment, the conjugate of the invention reduces plasma L-asparagine levels to undetectable levels for a time period of at least about 12, 24, 48, 72, 96, 120, 144, 168, 192, 216, or 240 hours when administered at a dose ranging from about 100 to about 15,000 IU/m² (about 1-30 mg protein/m²). In a particular embodiment, the conjugate comprises L-asparaginase from Erwinia species, more specifically Erwinia chrysanthemi, and more specifically, the L-asparaginase comprising the sequence of SEQ ID NO: 1. In a particular embodiment, the conjugate comprises PEG (e.g., mPEG) having a molecular weight of less than or equal to about 5000 Da. In a more particular embodiment, at least about 40% to about 100% of accessible amino groups (e.g., lysine residues and/or the N-terminus) are PEGylated.

In one embodiment, the conjugate comprises a ratio of mol PEG/mol monomer of about 4.5 to about 8.5, particularly about 6.5; a specific activity of about 450 to about 550 U/mg, particularly about 501 U/mg; and a relative activity of about 75% to about 85%, particularly about 81% compared to the corresponding unmodified L-asparaginase. In a specific embodiment, the conjugate with these properties comprises an L-asparaginase from Erwinia species, more specifically Erwinia chrysanthemi, and more specifically, the L-asparaginase comprising the sequence of SEQ ID NO: 1, with PEGylation of approximately 40-55% accessible amino groups (e.g., lysine residues and/or the N-terminus) with 5000 Da mPEG.

In one embodiment, the conjugate comprises a ratio of mol PEG/mol monomer of about 12.0 to about 18.0, particularly about 15.1; a specific activity of about 450 to about 550 U/mg, particularly about 483 U/mg; and a relative activity of about 75 to about 85%, particularly about 78% compared to the corresponding unmodified L-asparaginase. In a specific embodiment, the conjugate with these properties comprises an L-asparaginase from Erwinia species, more specifically Erwinia chrysanthemi, and more specifically, the L-asparaginase comprising the sequence of SEQ ID NO: 1, with PEGylation of approximately 100% accessible amino groups (e.g., lysine residues and/or the N-terminus) with 5000 Da mPEG.

In one embodiment, the conjugate comprises a ratio of mol PEG/mol monomer of about 5.0 to about 9.0, particularly about 7.0; a specific activity of about 450 to about 550 U/mg, particularly about 501 U/mg; and a relative activity of about 80 to about 90%, particularly about 87% compared to the corresponding unmodified L-asparaginase. In a specific embodiment, the conjugate with these properties comprises an L-asparaginase from Erwinia species, more specifically Erwinia chrysanthemi, and more specifically, the L-asparaginase comprising the sequence of SEQ ID NO: 1, with PEGylation of approximately 40-55% accessible amino groups (e.g., lysine residues and/or the N-terminus) with 10,000 Da mPEG.

In one embodiment, the conjugate comprises a ratio of mol PEG/mol monomer of about 11.0 to about 17.0, particularly about 14.1; a specific activity of about 450 to about 550 U/mg, particularly about 541 U/mg; and a relative activity of about 80 to about 90%, particularly about 87% compared to the corresponding unmodified L-asparaginase. In a specific embodiment, the conjugate with these properties comprises an L-asparaginase from Erwinia species, more specifically Erwinia chrysanthemi, and more specifically, the L-asparaginase comprising the sequence of SEQ ID NO: 1, with PEGylation of approximately 100% accessible amino groups (e.g., lysine residues and/or the N-terminus) with 10,000 Da mPEG.

In one embodiment, the conjugate comprises a ratio of mol PEG/mol monomer of about 6.5 to about 10.5, particularly about 8.5; a specific activity of about 450 to about 550 U/mg, particularly about 524 U/mg; and a relative activity of about 80 to about 90%, particularly about 84% compared to the corresponding unmodified L-asparaginase. In a specific embodiment, the conjugate with these properties comprises an L-asparaginase from Erwinia species, more specifically Erwinia chrysanthemi, and more specifically, the L-asparaginase comprising the sequence of SEQ ID NO: 1, with PEGylation of approximately 40-55% accessible amino groups (e.g., lysine residues and/or the N-terminus) with 2,000 Da mPEG.

In one embodiment, the conjugate comprises a ratio of mol PEG/mol monomer of about 12.5 to about 18.5, particularly about 15.5; a specific activity of about 450 to about 550 U/mg, particularly about 515 U/mg; and a relative activity of about 80 to about 90%, particularly about 83% compared to the corresponding unmodified L-asparaginase. In a specific embodiment, the conjugate with these properties comprises an L-asparaginase from Erwinia species, more specifically Erwinia chrysanthemi, and more specifically, the L-asparaginase comprising the sequence of SEQ ID NO: 1, with PEGylation of approximately 100% accessible amino groups (e.g., lysine residues and/or the N-terminus) with 2,000 Da mPEG.

In other embodiments, the conjugate of the invention has an increased potency of at least about 10 times, 20 times, 30 times, 40 times, 50 times, 60 times, 70 times, 80 times, 90 times, or 100 times after a single injection compared to the corresponding unmodified L-asparaginase. In a specific embodiment, the conjugate with these properties comprises an L-asparaginase from Erwinia species, more specifically Erwinia chrysanthemi, and more specifically, the L-asparaginase comprising the sequence of SEQ ID NO: 1. In a particular embodiment, the conjugate comprises PEG (e.g., mPEG) having a molecular weight of less than or equal to about 5000 Da. In a more particular embodiment, at least about 40% to about 100% of accessible amino groups (e.g., lysine residues and/or the N-terminus) are PEGylated.

In one embodiment, the conjugate of the invention has a single-dose pharmacokinetic profile determine as set forth in PCT Publication No. WO2011003886 according to the following, specifically wherein the conjugate comprises mPEG at molecular weight of less than or equal to 2000 Da and an L-asparaginase from Erwinia species, more specifically Erwinia chrysanthemi, and more specifically, the L-asparaginase comprising the sequence of SEQ ID NO: 1:

Aₘₐₓ: about 150 U/L to about 250 U/L;

T_{Amax}: about 4 h to about 8 h, specifically about 6 h;

d_{Amax}: about 220 h to about 250 h, specifically, about 238.5 h (above zero, from about 90 min to about 240 h);

AUC: about 12000 to about 30000; and

t½: about 50 h to about 90 h.

In one embodiment, the conjugate of the invention has a single-dose pharmacokinetic profile according to the following, specifically where the conjugate comprises mPEG at molecular weight of less than or equal to 5000 Da and an L-asparaginase from Erwinia species, more specifically Erwinia chrysanthemi, and more specifically, the L-asparaginase comprising the sequence of SEQ ID NO: 1:

Aₘₐₓ: about 18 U/L to about 250 U/L;

T_{Amax}: about 1 h to about 50 h;

d_{Amax}: about 90 h to about 250 h, specifically, about 238.5 h (above zero, from about 90 min to about 240 h);

AUC: about 500 to about 35000; and

t½: about 30 h to about 120 h.

In one embodiment, the conjugate of the invention results in a similar level of L-asparagine depletion over a period of time (e.g., 24, 48, or 72 hours) after a single dose compared to an equivalent quantity of protein of pegaspargase. In a specific embodiment, the conjugate comprises an L-asparaginase from Erwinia species, more specifically Erwinia chrysanthemi, and more specifically, the L-asparaginase comprising the sequence of SEQ ID NO: 1. In a particular embodiment, the conjugate comprises PEG (e.g., mPEG) having a molecular weight of less than or equal to about 5000 Da. In a more particular embodiment, at least about 40% to about 100% of accessible amino groups (e.g., lysine residues and/or the N-terminus) are PEGylated, more particularly about 40-55% or 100%.

In one embodiment, the conjugate of the invention has a longer t½ than pegaspargase administered at an equivalent protein dose. In a specific embodiment, the conjugate has a t½ of at least about 50, 52, 54, 56, 58, 59, 60, 61, 62, 63, 64, or 65 hours at a dose of about 50 μg/kg (protein content basis). In another specific embodiment, the conjugate has a t½ of at least about 30, 32, 34, 36, 37, 38, 39, or 40 hours at a dose of about 10 μg/kg (protein content basis). In another specific embodiment, the conjugate has a t½ of at least about 100 to about 200 hours at a dose ranging from about 100 to about 15,000 IU/m² (about 1-30 mg protein/m²).

In one embodiment, the conjugate of the invention has a mean AUC that is at least about 2, 3, 4 or 5 times greater than pegaspargase at an equivalent protein dose.

In one embodiment, the conjugate of the invention does not raise any significant antibody response for a particular period of time after administration of a single dose, e.g, greater than about 1 week, 2 weeks, 3 weeks, 4, weeks, 5 weeks, 6 weeks, 7 weeks, 8 weeks, 9 weeks, 10 weeks, 11 weeks, 12 weeks, etc. In a particular embodiment the conjugate of the invention does not raise any significant antibody response for at least 8 weeks. In one example, “does not raise any significant antibody response” means that the subject receiving the conjugate is identified within art-recognized parameters as antibody-negative. Antibody levels can be determined by methods known in the art, for example ELISA or surface plasmon resonance (SPR-Biacore) assays (Zalewska-Szewczyk (2009) Clin. Exp. Med. 9,113-116; Avramis (2009) Anticancer Research 29, 299-302 each of which is incorporated herein by reference in its entirety). Conjugates of the invention may have any combination of these properties.

### PASylated L-asparaginase

In some embodiments, the methods of the invention encompass a conjugate of L-asparaginase which comprises one or more peptide(s), wherein each is independently a peptide R^{N}-(P/A)-R^{C}, wherein (P/A) is an amino acid sequence consisting solely of proline and alanine amino acid residues, wherein R^{N} is a protecting group attached to the N-terminal amino group of the amino acid sequence, and wherein R^{C} is an amino acid residue bound via its amino group to the C-terminal carboxy group of the amino acid sequence, wherein each peptide is conjugated to the L-asparaginase via an amide linkage formed from the carboxy group of the C-terminal amino acid residue R^{C} of the peptide and a free amino group of the L-asparaginase, and wherein at least one of the free amino groups, which the peptides are conjugated to, is not an N-terminal α-amino group of the L-asparaginase. These molecules are also known as PASylated versions of L-asparaginase and are also referred to herein as conjugates.

The monomer of the modified L-asparaginase protein has from about 350, 400, 450, 500, amino acids to about 550, 600, 650, 700, or 750 amino acids after modification. In additional aspects, the modified L-asparaginase protein has from about 350 to about 750 amino acids, or about 500 to about 750 amino acids.

Each peptide that is comprised in the modified L-asparaginase protein as described herein is independently a peptide R^{N}-(P/A)-R^{C}. Accordingly, for each of the peptides comprised in a modified L-asparaginase protein described herein, the N-terminal protecting group R^{N}, the amino acid sequence (P/A), and the C-terminal amino acid residue R^{C} are each independently selected from their respective meanings. The two or more peptides comprised in the modified L-asparaginase protein may thus be the same, or they may be different from one another. In one aspect, all of the peptides comprised in the modified L-asparaginase protein are the same.

The moiety (P/A) in the chemically conjugated modified L-asparaginase protein, which is comprised in the peptide R^{N}-(P/A)-R^{C}, is an amino acid sequence that can consist of a total of between 10 to 100 or more proline and alanine amino acid residues, a total of 15 to 60 proline and alanine amino acid residues, a total of 15 to 45 proline and alanine amino acid residues, e.g. a total of 20 to about 40 proline and alanine amino acid residues, e.g. 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 proline and alanine amino acid residues. In a preferred aspect, said amino acid sequence consists of about 20 proline and alanine amino acid residues. In another preferred aspect, said amino acid sequence consists of about 40 proline and alanine amino acid residues. In the peptide R^{N}-(P/A)-R^{C}, the ratio of the number of proline residues comprised in the moiety (P/A) to the total number of amino acid residues comprised in (P/A) is preferably ≥10% and ≤70%, more preferably ≥20% and ≤50%, and even more preferably ≥25% and ≤40%. Accordingly, it is preferred that 10% to 70% of the total number of amino acid residues in (P/A) are proline residues; more preferably, 20% to 50% of the total number of amino acid residues comprised in (P/A) are proline residues; and even more preferably, 25% to 40% (e.g., 25%, 30%, 35% or 40%) of the total number of amino acid residues comprised in (P/A) are proline residues. Moreover, it is preferred that (P/A) does not contain any consecutive proline residues (i.e., that it does not contain any partial sequence PP). In a preferred aspect, (P/A) is the amino acid sequence AAPAAPAPAAPAAPAPAAPA (SEQ ID NO: 2). In another preferred aspect, (P/A) is the amino acid sequence

(SEQ ID NO: 3)
AAPAAPAPAAPAAPAPAAPAAAPAAPAPAAPAAPAPAAPA.

The group R^{N} in the peptide R^{N}-(P/A)-R^{C} may be a protecting group which is attached to the N-terminal amino group, particularly the N-terminal α-amino group, of the amino acid sequence (P/A). It is preferred that R^{N} is pyroglutamoyl or acetyl.

The group R^{C} in the peptide R^{N}-(P/A)-R^{C} is an amino acid residue which is bound via its amino group to the C-terminal carboxy group of (P/A), and which comprises at least two carbon atoms between its amino group and its carboxy group. It will be understood that the at least two carbon atoms between the amino group and the carboxy group of R^{C} may provide a distance of at least two carbon atoms between the amino group and the carboxy group of R^{C} (which is the case if, e.g., R^{C} is an ω-amino-C₃₋₁₅ alkanoic acid, such as ε-aminohexanoic acid). It is preferred that R^{C} is ε-aminohexanoic acid.

In one embodiment, the peptide is Pga-AAPAAPAPAAPAAPAPAAPA-Ahx-COOH (SEQ ID NO: 4) or Pga-AAPAAPAPAAPAAPAPAAPAAAPAAPAPAAPAAPAPAAPA-Ahx-COOH (SEQ ID NO: 5). The term “Pga” is an abbreviation of “pyroglutamoyl” or “pyroglutamic acid”. The term “Ahx” is an abbreviation of “ε-aminohexanoic acid”.

In the modified L-asparaginase proteins as described herein, each peptide R^{N}-(P/A)-R^{C}, can be conjugated to the L-asparaginase via an amide linkage formed from the carboxy group of the C-terminal amino acid residue R^{C} of the peptide and a free amino group of the L-asparaginase. A free amino group of the L-asparaginase may be, e.g., an N-terminal α-amino group or a side-chain amino group of the L-asparaginase (e.g., an ε-amino group of a lysine residue comprised in the L-asparaginase). If the L-asparaginase is composed of multiple subunits, e.g. if the L-asparaginase is a tetramer, there may be multiple N-terminal α-amino groups (i.e., one on each subunit). In one aspect, 9 to 13 peptides as defined herein (e.g. 9, 11, 12, or 13 peptides) can be chemically conjugated to the L-asparaginase (e.g. to each subunit/monomer of the L-asparaginase).

In accordance with the above, in one aspect at least one of the free amino groups, which the peptides are chemically conjugated to, is not (i.e., is different from) an N-terminal α-amino group of the L-asparaginase. Accordingly, it is preferred that at least one of the free amino groups, which the peptides are conjugated to, is a side-chain amino group of the L-asparaginase, and it is particularly preferred that at least one of the free amino groups, which the peptides are conjugated to, is an ε-amino group of a lysine residue of the L-asparaginase.

Moreover, it is preferred that the free amino groups, which the peptides are conjugated to, are selected from the ε-amino group(s) of any lysine residue(s) of the L-asparaginase, the N-terminal α-amino group(s) of the L-asparaginase or of any subunit(s) of the L-asparaginase, and any combination thereof. It is particularly preferred that one of the free amino groups, which the peptides are conjugated to, is an N-terminal α-amino group, while the other one(s) of the free amino groups, which the peptides are conjugated to, is/are each an ε-amino group of a lysine residue of the L-asparaginase. Alternatively, it is preferred that each of the free amino groups, which the peptides are conjugated to, is an ε-amino group of a lysine residue of the L-asparaginase.

The modified L-asparaginase proteins as described herein are composed of L-asparaginase and one or more peptides as defined herein. A corresponding modified L-asparaginase protein may, e.g., consist of one L-asparaginase and one, two, three, four, five, six, seven, eight, nine, ten, 15, 20, 25, 30, 35, 40, 45, 50, 55 (or more) peptides which are each conjugated to the L-asparaginase. The L-asparaginase may be, e.g., a monomeric protein or a protein composed of multiple subunits, e.g. a tetramer. If the L-asparaginase is a monomeric protein, a corresponding modified L-asparaginase protein may, e.g., consist of one monomeric L-asparaginase and nine to thirteen (or more) (e.g., 8, 9, 10, 11, 12, or 13), peptides which are each conjugated to the monomeric L-asparaginase. An exemplary amino acid sequence of a monomeric L-asparaginase is shown in SEQ ID NO: 1. If the L-asparaginase is a protein composed of multiple subunits, e.g. of four subunits (i.e. if said L-asparaginase is a tetramer), a corresponding modified L-asparaginase protein may, e.g., consist of four L-asparaginase subunits and nine to thirteen (or more) (e.g. 9, 10, 11, 12, or 13), peptides as defined herein which are each conjugated to each subunit of the L-asparaginase. An exemplary amino acid sequence of a subunit of L-asparaginase is shown in SEQ ID NO. 1. Likewise, if the L-asparaginase is a protein composed of multiple subunits, e.g. of four subunits (i.e. if said L-asparaginase is a tetramer), a corresponding modified L-asparaginase protein may, e.g., consist of four L-asparaginase subunits and 30, 31, 32, 33, 34, 35, 36, 37, 38, 39, 40, 41, 42, 43, 44, 45, 46, 47, 48, 49, 50, 51, 52, 53, 54, 55 (or more) peptides which are each conjugated to the L-asparaginase tetramer. In one aspect the invention relates to a modified L-asparaginase protein having an L-asparaginase and multiple chemically attached peptide sequences. In a further aspect the length of the peptide sequences are from about 10 to about 100, from about 15 to about 60 or from about 20 to about 40.

The peptide consisting solely of proline and alanine amino acid residues may be covalently linked to one or more amino acids of said L-asparaginase, such as lysine residues and/or N-terminal residue, and/or the peptide consisting solely of proline and alanine amino acid residues may be covalently linked to at least from about 40, 50, 60, 70, 80 or 90% to about 60, 70, 80, 90 or 100% of the accessible amino groups including amino groups of lysine residues and/or N-terminal residue on the surface of the L-asparaginase. For example, there may be about 11 to 12 lysine residues accessible per L-asparaginase, and about 8 to 12 lysines would be conjugated to the peptide consisting solely of proline and alanine amino acid residues. In further aspects, the peptide consisting solely of proline and alanine amino acid residues is covalently linked to from about 20, 30, 40, 50, or 60% to about 30, 40, 50, 60, 70, 80 or 90% of total lysine residues of said L-asparaginase. In further embodiments, the peptide consisting solely of proline and alanine amino acid residues is covalently linked to the L-asparaginase via a linker. Exemplary linkers include linkers disclosed in U.S. Patent Application No. 2015/0037359, which is herein incorporated by reference in its entirety.

In one aspect, the conjugate is a fusion protein comprising L-asparaginase and a polypeptide consisting solely of proline and alanine amino acid residues of a length of about 200 to about 400 proline and alanine amino acid residues. In other words the polypeptide may consist of about 200 to about 400 proline and alanine amino acid residues. In one aspect, the polypeptide consists of a total of about 200 (e.g. 201) proline and alanine amino acid residues (i.e. has a length of about 200 (e.g. 201) proline and alanine amino acid residues) or the polypeptide consists of a total of about 400 (e.g. 401) proline and alanine amino acid residues (i.e. has a length of about 400 (e.g. 401) proline and alanine amino acid residues). In some preferred embodiments, the polypeptide comprises or consists of an amino acid sequence as shown in SEQ ID NO: 6 or 7. In some aspects, the fusion protein each monomer has from about 350, 400, 450, 500, amino acids to about 550, 600, 650, 700, 750 or 1,000 amino acids including the monomer and the P/A amino acid sequence. In additional aspects, the modified protein has from about 350 to about 800 amino acids or about 500 to about 750 amino acids. For example, the polypeptide includes the peptides prepared in U.S. Pat. No. 9,221,882. In some aspects, the L-asparaginase is from an Erwinia species, more specifically Erwinia chrysanthemi, and more specifically, the L-asparaginase comprising the sequence of SEQ ID NO: 1 as described herein.

In additional aspects, the L-asparaginase disclosed herein can be produced using a (recombinant) vector comprising the nucleotide sequence encoding the modified L-asaparaginase protein comprising the L-asparaginase and a polypeptide, wherein the polypeptide consists solely of proline and alanine amino acid residues, preferably wherein the modified protein is a fusion protein, as described herein, wherein the vector can express the modified protein (e.g. fusion protein). In further aspects, the invention also relates to a host comprising the (recombinant) vector described herein. The host may be yeasts, such as Saccharomyces cerevisiae and Pichia Pistoris, bacteria, actinomycetes, fungi, algae, and other microorganisms, including Escherichia coli, Bacillus sp., Pseudomonas fluorescens, Corynebacterium glutamicum and bacterial hosts of the following genuses, Serratia, Proteus, Acinetobacter and Alcaligenes. Other hosts are known to those of skill in the art, including Nocardiopsis alba, which expresses a variant of Asparaginase lacking on glutaminase-activity, and those disclosed in Savitri et al. (2003) Indian Journal of Biotechnology, 2, 184-194, which is incorporated by reference herein in its entirety.

### Methods of Treatment and Use

The conjugates s of the invention can be used in the treatment of a disease treatable by depletion of asparagine and/or glutamine. For example, the conjugate is useful in the treatment or the manufacture of a medicament for use in the treatment of acute lymphoblastic Leukemia (ALL) in both adults and children, as well as other conditions where asparagine and/or glutamine depletion is expected to have a useful effect. Such conditions include, but are not limited to the following: malignancies, or cancers, including but not limited to hematologic malignancies, lymphoma, large cell immunoblastic lymphoma, non-Hodgkin's lymphoma, diffuse large B-cell lymphoma, NK lymphoma, Hodgkin's disease, acute myelocytic Leukemia, acute promyelocytic Leukemia, acute myelomonocytic Leukemia, acute monocytic Leukemia, acute T-cell Leukemia, acute myeloid Leukemia (AML), biphenotypic B-cell myelomonocytic Leukemia, chronic lymphocytic Leukemia, lymphosarcoma, reticulosarcoma, and melanosarcoma. In some embodiments, the disease may be acute myeloid leukemia or diffuse large B-cell lymphoma. Malignancies or cancers, include but not limited to, renal cell carcinoma, renal cell adenocarcinoma, glioblastoma including glioblastoma multiforma and glioblastoma astrocytoma, medulloblastoma, rhabdomyosarcoma, malignant melanoma, epidermoid carcinoma, squamous cell carcinoma, lung carcinoma including large cell lung carcinoma and small cell lung carcinoma, endometrial carcinoma, ovarian adenocarcinoma, ovarian tetratocarcinoma, cervical adenocarcinoma, breast carcinoma, breast adenocarcinoma, breast ductal carcinoma, pancreatic adenocarcinoma, pancreatic ductal carcinoma, colon carcinoma, colon adenocarcinoma, colorectal adenocarcinoma, bladder transitional cell carcinoma, bladder papilloma, prostate carcinoma, osteosarcoma, epitheloid carcinoma of the bone, prostate carcinoma, and thyroid cancer.

Representative non-malignant hematologic diseases which respond to asparagine and/or glutamine depletion include immune system-mediated Blood diseases, e.g., infectious diseases such as those caused by HIV infection (i.e., AIDS). Non-hematologic diseases associated with asparagine and/or glutamine dependence include autoimmune diseases, for example rheumatoid arthritis, systemic lupus erythematosus (SLE), collagen vascular diseases, etc. Other autoimmune diseases include osteo-arthritis, Issac's syndrome, psoriasis, insulin dependent diabetes mellitus, multiple sclerosis, sclerosing panencephalitis, rheumatic fever, inflammatory bowel disease (e.g., ulcerative colitis and Crohn's disease), primary billiary cirrhosis, chronic active hepatitis, glomerulonephritis, myasthenia gravis, pemphigus vulgaris, and Graves' disease. Cells suspected of causing disease can be tested for asparagine and/or glutamine dependence in any suitable in vitro or in vivo assay, e.g., an in vitro assay wherein the growth medium lacks asparagine and/or glutamine. Thus, in one aspect, the invention is directed to a method of treating a disease treatable in a patient, the method comprising administering to the patient an effective amount of a conjugate of the invention. In another aspect, the conjugate of the invention is co-administered with another active pharmaceutical ingredient. In some embodiments, the conjugate of the invention is co-administered with Oncaspar®, daunorubicin, cytarabine, Vyxeos®, ABT-737, Venetoclax, dactolisib, bortezomib, carfilzomib, vincristine, prednisolone, everolimus, and/or CB-839. In a specific embodiment, the disease is ALL. In a particular embodiment, the conjugate used in the treatment of a disease treatable by asparagine and/or glutamine depletion comprises an L-asparaginase from Erwinia species, more specifically Erwinia chrysanthemi, and more specifically, the L-asparaginase comprising the sequence of SEQ ID NO: 1 as described herein.

In one embodiment, treatment with a conjugate of the invention will be administered as a first line therapy. In another embodiment, treatment with a conjugate of the invention will be administered as a second line therapy in patients, particularly patients with ALL, where objective signs of allergy or hypersensitivity, including “silent hypersensitivity,” have developed to other asparaginase preparations, in particular, the native Escherichia-coli-derived L-asparaginase or its PEGylated variant (pegaspargase). Non-limiting examples of objective signs of allergy or hypersensitivity include testing “antibody positive” for an asparaginase enzyme. In a specific embodiment, the conjugate of the invention is used in second line therapy after treatment with pegaspargase. In a more specific embodiment, the conjugate used in second line therapy comprises an L-asparaginase from Erwinia species, more specifically Erwinia chrysanthemi, and more specifically, the L-asparaginase comprising the sequence of SEQ ID NO: 1. In a more specific embodiment, the conjugate further comprises PEG (e.g., mPEG) having a molecular weight of less than or equal to about 5000 Da, more specifically about 5000 Da. In an even more specific embodiment, at least about 40% to about 100% of accessible amino groups (e.g., lysine residues and/or the N-terminus) are PEGylated, more particularly about 40-55% or 100%.

In another aspect, the invention is directed to a method for treating acute lymphoblastic leukemia comprising administering to a patient in need of the treatment a therapeutically effective amount of a conjugate of the invention. In another aspect, the invention is directed to a method for treating acute myeloid leukemia comprising co-administering to a patient in need of the treatment a therapeutically effective amount of a conjugate of the invention in combination with daunorubicin, cytarabine, Vyxeos®, ABT-737, venetoclax, dactolisib, bortexomib, and/or carfilzomib. In another aspect, the invention is directed to a method for treating acute myeloid leukemia comprising co-administering to a patient in need of the treatment a therapeutically effective amount of a conjugate of the invention in combination with venetoclax. In another aspect, the invention is directed to a method for treating diffuse large B-cell lymphoma comprising co-administering to a patient in need of the treatment a therapeutically effective amount of a conjugate of the invention in combination with ABT-737, venetoclax, carfilzomib, vincristine, and/or prednisolone. In another aspect, the invention is directed to a method for treating diffuse large B-cell lymphoma comprising co-administering to a patient in need of the treatment a therapeutically effective amount of a conjugate of the invention in combination with vincristine.

In another aspect, the conjugate described herein will be administered at a dose ranging from about 1500 IU/m² to about 15,000 IU/m², typically about 10,000 to about 15,000 IU/m² (about 20-30 mg protein/m²), at a schedule ranging from about twice a week to about once a month, typically once per week or once every other week, as a single agent (e.g., monotherapy) or as part of a combination of chemotherapy drugs, including, but not limited to glucocorticoids, corticostcroids, anticancer compounds or other agents, including, but not limited to methotrexate, dexamethasone, prednisone, prednisolone, vincristine, cyclophosphamide, and anthracycline. As an example, patients with ALL will be administered the conjugate of the invention as a component of multi-agent chemotherapy during chemotherapy phases including induction, consolidation or intensification, and maintenance. In a specific example, the conjugate is not administered with an asparagine synthetase inhibitor (e.g., such as set forth in U.S. Pat. No. 9,920,311 which is herein incorporated by reference in its entirety). In another specific example, the conjugate is not administered with an asparagine synthetase inhibitor, but is administered with other chemotherapy drugs. The conjugate can be administered before, after, or simultaneously with other compounds as part of a multi-agent chemotherapy regimen.

In a specific embodiment, the method comprises administering a conjugate of the invention at an amount of about 1 U/kg to about 25 U/kg (e.g., about 1, 2, 3, 4, 5, 6, 7, 8, 9, 10, 11, 12, 13, 14, 15, 16, 17, 18, 19, 20, 21, 22, 23, 24, or 25 U/kg) or an equivalent amount thereof 20 (e.g., on a protein content basis). In a more specific embodiment, the conjugate is administered at an amount selected from the group consisting of about 5, about 10, and about 25 U/kg. In another specific embodiment, the conjugate is administered at a dose ranging from about 1,000 IU/m² to about 20,000 IU/m² (e.g., 1,000 IU/m², 2,000 IU/m², 3,000 IU/m², 4,000 IU/m², 5,000 IU/m², 6,000 IU/m², 7,000 IU/m², 8,000 IU/m², 9,000 IU/m², 10,000 IU/m², 11,000 IU/m², 12,000 IU/m², 13,000 IU/m², 14,000 IU/m², 15,000 IU/m², 16,000 IU/m², 17,000 IU/m², 18,000 IU/m², 19,000 IU/m², or 20,000 IU/m²). In another specific embodiment, the conjugate is administered at a dose that depletes L-asparagine and/or glutamine to undetectable levels using methods and apparatus known in the art for a period of about 3 days to about 10 days (e.g., 3, 4, 5, 6, 7, 8, 9, or 10 days) for a single dose.

In another embodiment, the method comprises administering a conjugate of the invention that elicits a lower immunogenic response in a patient compared to an unconjugated L-asparaginase. In another embodiment, the method comprises administering a conjugate of the invention that has a longer in vivo circulating half-life after a single dose compared to the unconjugated L-asparaginase. In one embodiment, the method comprises administering a conjugate that has a longer t½ than pegaspargase administered at an equivalent protein dose. In a specific embodiment, the method comprises administering a conjugate that has a t½ of at least about 50, 52, 54, 56, 58, 59, 60, 61, 62, 63, 64, or 65 hours at a dose of about 50 μg/kg (protein content basis). In another specific embodiment, the method comprises administering a conjugate that has a t½ of at least about 30, 32, 34, 36, 37, 37, 39, or 40 hours at a dose of about 10 μg/kg (protein content basis). In another specific embodiment, the method comprises administering a conjugate that has a t½ at least about 100 to about 200 hours at a dose ranging from about 10,000 to about 15,000 IU/IU/m² (about 20-30 mg protein/IU/m²). In one embodiment, the method comprises administering a conjugate that has a mean AUC that is at least about 2, 3, 4 or 5 times greater than pegaspargase at an equivalent protein dose.

The incidence of relapse in ALL patients following treatment with L-asparaginase remains high, with approximately 10-25% of pediatric ALL patients having early relapse (e.g. some during maintenance phase at 30-36 month post-induction) (Avramis (2005) Clin. Pharmacokinet. 44, 367-393). If a patient treated with E. coli-derived L-asparaginase has a relapse, subsequent treatment with E. coli preparations could lead to a “vaccination” effect, whereby the E. coli preparation has increased immunogenicity during the subsequent administrations. In one embodiment, the conjugate of the invention may be used in a method of treating patients with relapsed ALL who were previously treated with other asparaginase preparations, in particular those who were previously treated with E. coli-derived asparaginases.

In some embodiments, the uses and methods of treatment of the invention comprise administering an L-asparaginase conjugate having properties or combinations of properties described herein above (e.g., in the section entitled L-asparaginase PEG conjugates or PASylated L-asparaginase) or herein below.

### Compositions, Formulations, and Routes of Administration

The invention also includes a pharmaceutical composition comprising a conjugate of the invention. In a specific embodiment, the pharmaceutical composition is contained in a vial as a lyophilized powder to be reconstituted with a solvent, such as currently available native L-asparaginases, whatever the bacterial source used for its production (Kidrolase®, Elspar®, Erwinase®). In another embodiment, the pharmaceutical composition may further comprises a “ready to use” solution, such as pegaspargase (Oncaspar®) enabling, further to an appropriate handling, an administration through, e.g., intramuscular, intravenous (infusion and/or bolus), intra-cerebro-ventricular (icy), subcutaneous routes. In additional embodiments, the pharmaceutical composition may comprise the conjugate of the invention in combination with Oncaspar®, daunorubicin, cytarabine, ABT-737, Venetoclax, dactolisib, bortezomib, carfilzomib, vincristine, prednisolone, everolimus, and/or CB-839.

Conjugates of the invention, including compositions comprising conjugates of the invention (e.g., a pharmaceutical composition) can be administered to a patient using standard techniques. Techniques and formulations generally may be found in Remington's Pharmaceutical Sciences (2013) 22nd ed., Mack Publishing herein incorporated by reference.

Suitable dosage forms, in part, depend upon the use or the route of entry, for example, oral, transdermal, transmucosal, or by injection (parenteral). Such dosage forms should allow the therapeutic agent to reach a target cell or otherwise have the desired therapeutic effect. For example, pharmaceutical compositions injected into the Blood stream preferably are soluble.

Conjugates and/or pharmaceutical compositions according to the invention can be formulated as pharmaceutically acceptable salts and complexes thereof. Pharmaceutically acceptable salts are non-toxic salts present in the amounts and concentrations at which they are administered. The preparation of such salts can facilitate pharmaceutical use by altering the physical characteristics of the compound without preventing it from exerting its physiological effect. Useful alterations in physical properties include lowering the melting point to facilitate transmucosal administration and increasing solubility to facilitate administering higher concentrations of the drug. The pharmaceutically acceptable salt of an asparaginase may be present as a complex, as those in the art will appreciate.

Pharmaceutically acceptable salts include acid addition salts such as those containing sulfate, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate, and quinate. Pharmaceutically acceptable salts can be obtained from acids, including hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, supra. Such salts can be prepared using the appropriate corresponding bases.

Pharmaceutically acceptable carriers and/or excipients can also be incorporated into a pharmaceutical composition according to the invention to facilitate administration of the particular asparaginase. Examples of carriers suitable for use in the practice of the invention include calcium carbonate, calcium phosphate, various sugars such as lactose, glucose, or sucrose, or types of starch, cellulose derivatives, gelatin, vegetable oils, polyethylene glycols, and physiologically compatible solvents. Examples of physiologically compatible solvents include sterile solutions of water for injection (WFI), saline solution and dextrose.

Pharmaceutical compositions according to the invention can be administered by different routes, including intravenous, intraperitoneal, subcutaneous, intramuscular, oral, topical (transdermal), or transmucosal administration. For systemic administration, oral administration is preferred. For oral administration, for example, the compounds can be formulated into conventional oral dosage forms such as capsules, tablets, and liquid preparations such as syrups, elixirs, and concentrated drops.

Alternatively, injection (parenteral administration) may be used, e.g., intramuscular, intravenous, intraperitoneal, and subcutaneous injection. For injection, pharmaceutical compositions are formulated in liquid solutions, preferably in physiologically compatible buffers or solutions, such as saline solution, Hank's solution, or Ringer's solution. In addition, the compounds may be formulated in solid form and redissolved or suspended immediately prior to use. For example, lyophilized forms of the conjugate can be produced. In a specific embodiment, the conjugate is administered intramuscularly. In another specific embodiment, the conjugate is administered intravenously.

Systemic administration can also be accomplished by transmucosal or transdermal means. For transmucosal or transdermal administration, penetrants appropriate to the barrier to be permeated are used in the formulation. Such penetrants are well known in the art, and include, for example, for transmucosal administration, bile salts, and fusidic acid derivatives. In addition, detergents may be used to facilitate permeation. Transmucosal administration, for example, may be through nasal sprays, inhalers (for pulmonary delivery), rectal suppositories, or vaginal suppositories. For topical administration, compounds can be formulated into ointments, salves, gels, or creams, as is well known in the art.

The amounts of the conjugate to be delivered will depend on many factors, for example, the IC₅₀, EC₅₀, the biological half-life of the compound, the age, size, weight, and physical condition of the patient, and the disease or disorder to be treated. The importance of these and other factors to be considered are well known to those of ordinary skill in the art. Generally, the amount of the conjugate to be administered will range from about 10 International Units per square meter of the surface area of the patient's body (IU/m²) to 50,000 IU/m², with a dosage range of about 1,000 IU/m² to about 15,000 IU/m² being preferred, and a range of about 6,000 IU/m² to about 15,000 IU/ m² being more preferred, and a range of about 10,000 to about 15,000 IU/m² (about 20-30 mg protein/m²) being particularly preferred to treat a malignant hematologic disease, e.g., Leukemia. Typically, these dosages arc administered via intramuscular or intravenous injection at an interval of about 3 times weekly to about once per month, typically once per week or once every other week during the course of therapy. Of course, other dosages and/or treatment regimens may be employed, as determined by the attending physician.

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

### EXAMPLES

The subject matter of U.S. Pat. No. 9,920,311 is herein incorporated by reference including the Examples disclosing methods of producing and testing PEGylated Asparaginase. The mPEG-r-crisantaspase conjugate used in the following examples was prepared as set forth in U.S. Pat. No. 9,920,311.

### Example 1

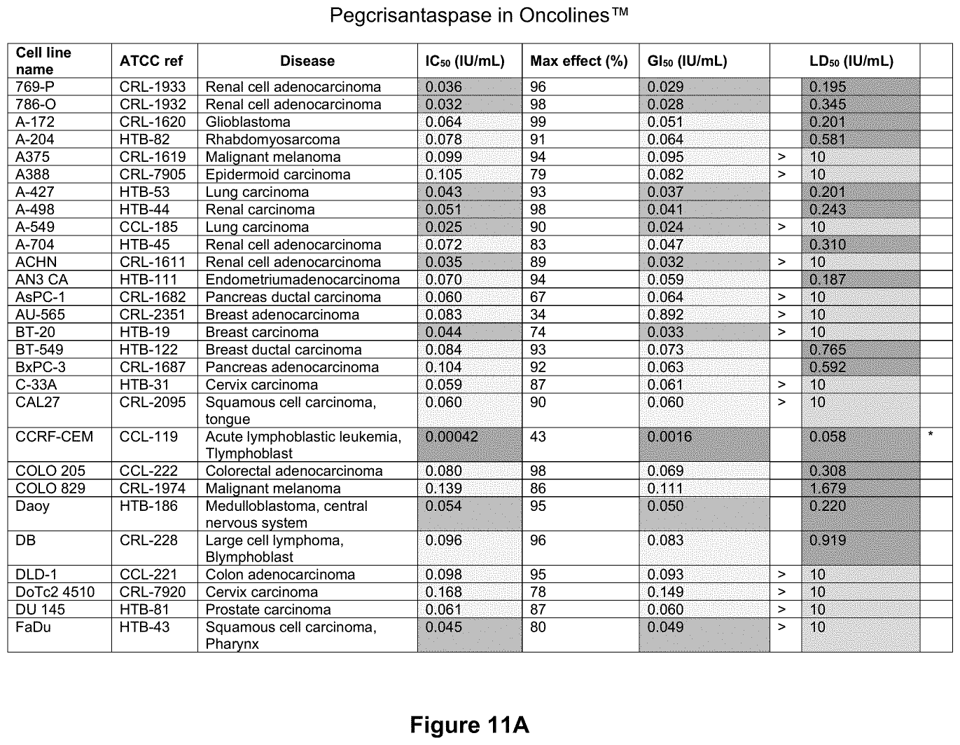

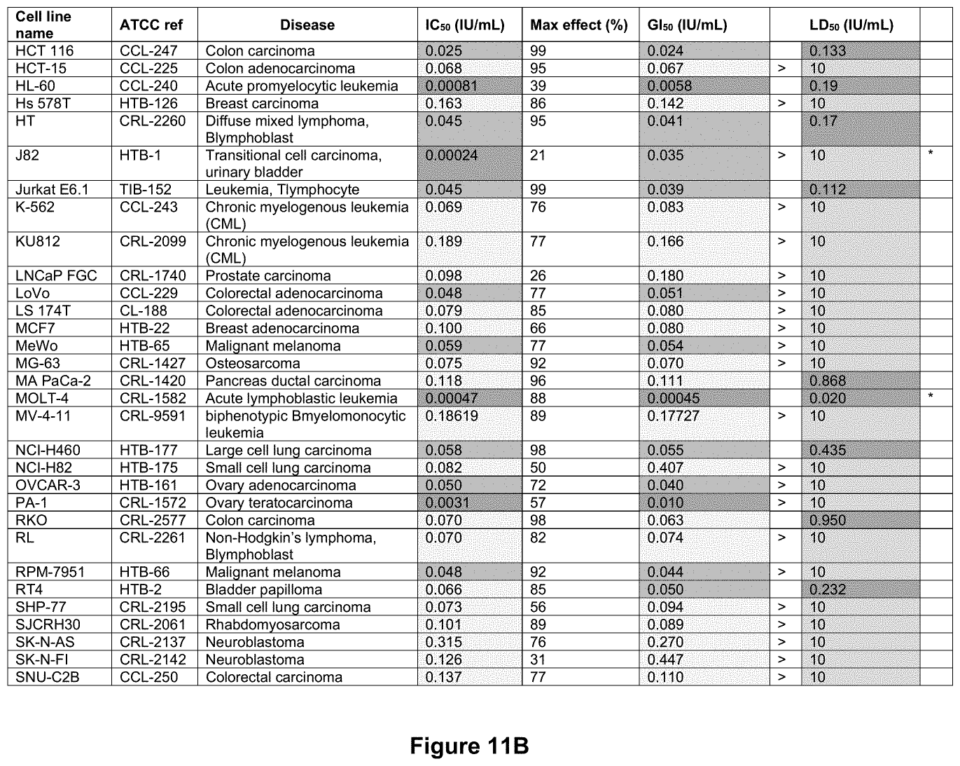

mPEG-r-crisantaspase conjugate (Pegcrisantaspase) was tested against various cell lines as shown in two stages in FIG. 11A.

Cell preparation. All cell lines have been licensed from the American Type Culture Collection (ATCC) Manassas, Va. (US). Master and Working Cell banks (MCB and WCB) were prepared by subculturing in ATCC-recommended media and freezing according to ATCC recommended protocols (www.atcc.org).

Compound preparation. Test compounds were prepared as stock solutions in DMSO or aqueous buffers as appropriate and serially diluted to obtain a dilution series.

Cell proliferation assay. Cell proliferation was assessed using a commercially available luminescence assay using ATP as the endpoint.

Controls. t=0 signal. On a parallel plate, 45 μl cells were dispensed and incubated in a humidified atmosphere of 5% CO2 at 37° C. After 24 hours 5 μl DMSO-containing Hepes buffer and 25 μl ATPlite 1Step™ solution were mixed, and luminescence measured after 10 minute incubation (=luminescencet=0).

Reference compound. The IC₅₀ of the reference compound doxorubicin is measured on a separate plate. The IC₅₀ is trended. If the IC₅₀ is out of specification (0.32-3.16 times deviating from historic average) the assay is invalidated.

Cell growth control. The cellular doubling times of all cell lines are calculated from the t=0 hours and t=end growth signals of the untreated cells. If the doubling time is out of specification (0.5-2.0 times deviating from historic average) the assay is invalidated.

Maximum signals. For each cell line, the maximum luminescence was recorded after incubation until t=end without compound in the presence of 0.4% DMSO (=luminescence_{untreated,t=end}).

Drug sensitivity. The ‘log IC₅₀ differences between the “modified and “wild type’ groups of cell lines were analyzed in three ways. First, for the eighteen most frequent genetic changes, drug sensitivities of individual cell lines were visualized in waterfall plots. Secondly, a larger subset of the most commonly occurring and best known cancer genes (38 in total) was analyzed with type II Anova analysis in the statistical program R. The results are displayed in a volcano plot. Thirdly, the complete set of 114 cancer genes was analyzed by a two-sided homoscedastic t-test in R. The p-values from Anova and t-test were subjected to a Benjamini-Hochberg multiple testing correction, and only genetic associations with a false discovery rate less than 20% are considered significant. The type II Anova analysis on 38 cancer genes is a different test than the homoscedastic t-test on 114 cancer genes, meaning that the significance of the associations may differ. For more information on Oncolines™ methods see www.ntrc.nl/services/oncolinestm.

IC₅₀ were calculated by non-linear regression using IDBS XLfit. The percentage growth after incubation until t=end (%-growth) was calculated as follows: 100%×(luminescence_{t=end}/luminescence_{untreated,t=end}). This was fitted to the ¹⁰log compound concentration (conc) by a 4-parameter logistics curve: %-growth=bottom+(top−bottom)/(1+10^{(logIC50-conc)*hill)}) where hill is the Hill-coefficient, and bottom and top the asymptotic minimum and maximum cell growth that the compound allows in that assay.

NCI60 parameters. The LD₅₀, the concentration at which 50% of cells die, is the concentration where luminescence_{t=end}=½×luminescenceₜ₌₀ₕ. The GI₅₀, the concentration of 50% growth inhibition, is the concentration where cell growth is half maximum. This is concentration associated with the signal: ((luminescence_{untreated,t=end}−luminescenceₜ₌₀)/2)+luminescenceₜ₌₀.

Curve fitting. Curves calculated automatically by the software were adjusted manually according to the following protocol: The curve bottom was fixed at 0% when the calculated curve had a bottom below zero. The hill was fixed on −6 when the software calculated a lower value. Curves were invalidated when the F-test value for fitting quality was >1.5 or when the compound was inactive (<20% maximal effect), in which cases curves were removed from the graphs. When a curve had a biphasic character, it was fitted on the most potent IC₅₀. Incidentally, when technical failures were likely, concentration points were knocked out. This is always shown in the dose-response graphs. The maximal effect (Max effect) was calculated as 100% (signal of untreated cells) minus the curve bottom when the dose-response curve was completely determined for more than 85%. A dose-response curve is considered 100% complete when the data points at the highest concentrations reach the curve bottom. If the completeness was smaller than 85%, Max effect was calculated as 100% minus the average of the lowest signal. In cases where the bottom of the curve was locked on 0%, the maximal effect was always calculated as 100% minus the growth inhibition at the highest concentration.

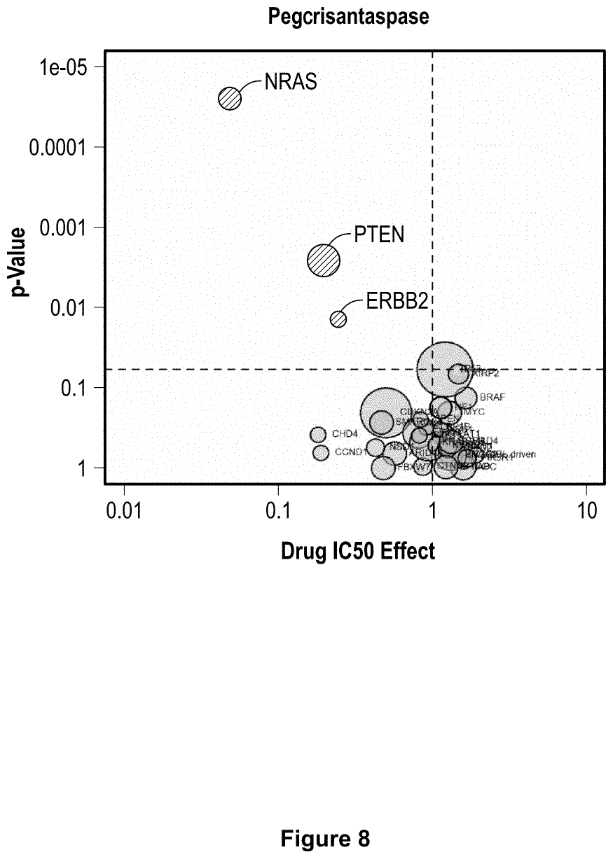

Volcano plot. The volcano plot in FIG. 8 shows how genetic transformations in 38 important genes are statistically associated with shifts in compound sensitivity (as measured by ¹⁰logIC₅₀). The p-value (y-axis in the volcano plot) indicates the confidence level for genetic association of mutations in a particular gene with a IC₅₀ shift. The factor of the IC₅₀ shift is indicated on the x-axis. The areas of the circles are proportional to the number of mutants in the cell panel (each mutation is present at least three times). To compute significance, p-values are subjected to a Benjamin-Hochberg multiple testing correction, and only genetic associations with a <20% false discovery rate are colored grey. The relevant cutoff p-value (0.059) is indicated by a horizontal line. If there are no significant associations, no grey circles and horizontal line are drawn.

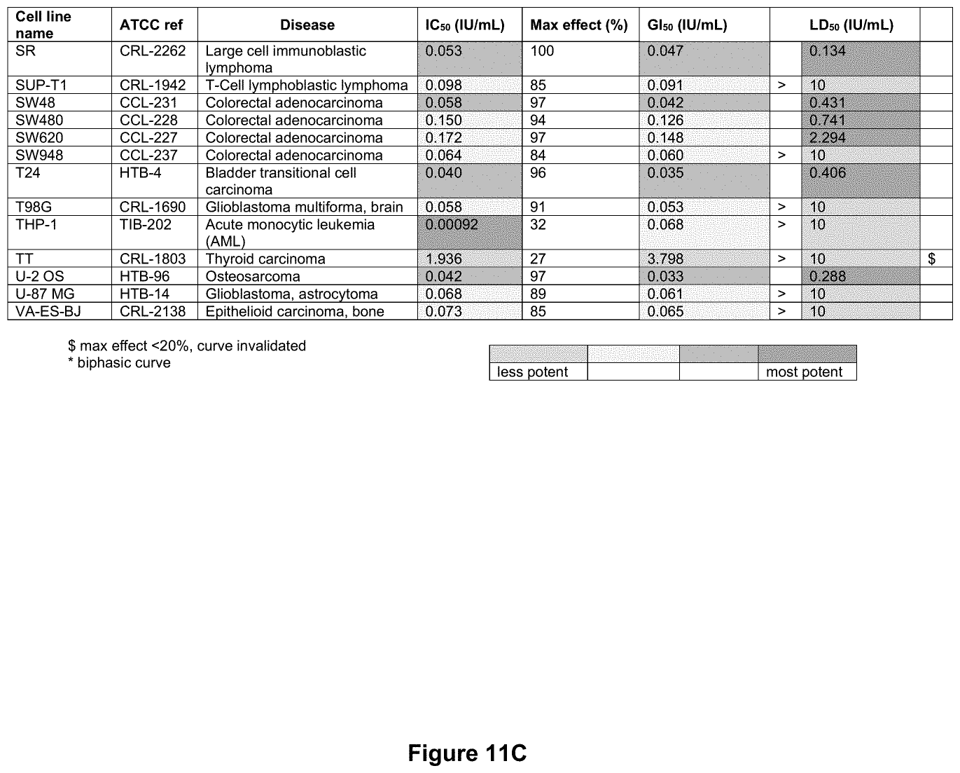

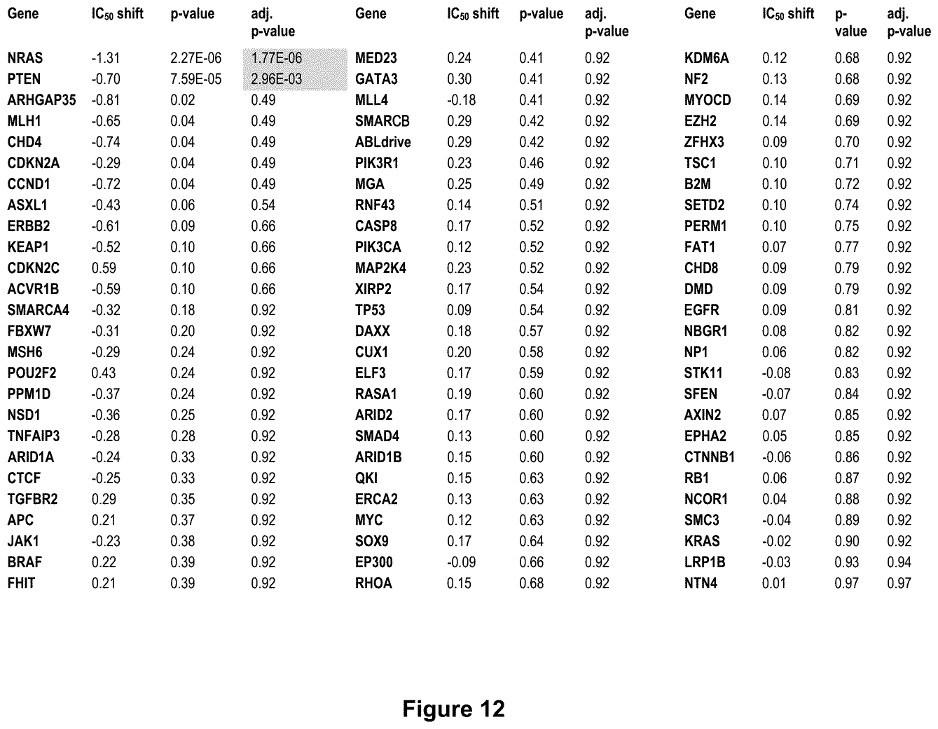

Results of the T-test. For 98 validated cancer driver genes, of which mutations also occur in patients, it was tested if presence of ‘wild type’ and ‘mutant’ variants of the gene in cell lines, is associated with a significant IC₅₀s shift of the investigated compound. The column ‘IC₅₀ shift’ indicates the ¹⁰logIC₅₀ difference. A negative IC₅₀ shift indicates that the compound is more potent in cell lines that carry the ‘mutant’ gene. The column ‘p-value’ indicates the result of a two-sided t-test. To compute significance, p-values were subjected to a Benjamin-Hochberg multiple testing correction, and only genetic associations with a <20% false discovery rate are highlighted (column ‘adj. p-value’). If there are no significant associations, there are no grey cells in the table depicted in FIG. 12.

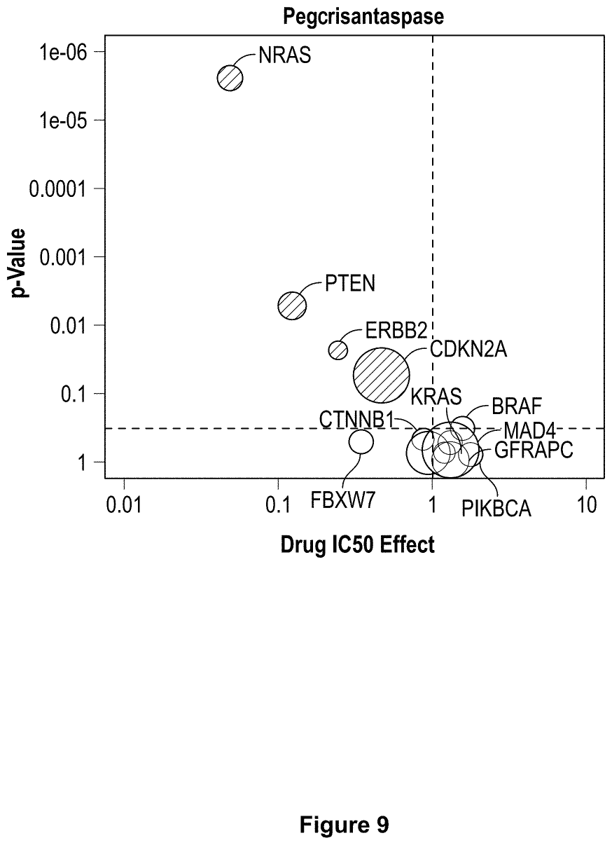

The special volcano plot of FIG. 9 relates compound sensitivity (as measured by ¹⁰logIC₅₀) to the presence of cancer hotspot mutations. This provides increased focus on clinically relevant cancer driver mutations in comparison to the previous analyses. The hotspot mutations were derived from statistical analyses of the recurrence patterns of mutations and copy number alterations in patients through separate studies. Axes and statistical analyses are identical to the volcano plot of FIG. 8. The cutoff p-level for significance is 0.32.
Example 3: Synergistic activity of Pegcrisantaspase and Oncaspar®. Effect₂₀ For determination of the effect of the compound on the activity of other anti-cancer agents in SynergyScreen™ experiments, a low, fixed concentration is used, corresponding to the concentration at which cell growth is inhibited by 20%. This concentration is determined using the dose-response curves of the single compounds. The concentration is the value on the x-as, corresponding to 80% viability of untreated at the y-axis.

Oncaspar in Oncolines

| Cell line | ATCC ref. | Disease | Effect₂₀ (IU/mL) |
|---|---|---|---|
| KG-1 | CCL-246 | Acute myelogenous leukemia (AML) | 0.0001 |
| HL-60 | CCL-240 | Acute promyelocytic leukemia | 0.0003 |
| THP-1 | TIB-202 | Acute monocytic leukemia | 0.31 |
| DB | CRL-2289 | Large cell lymphoma, B lymphoblast | 0.47 |
| HT | CRL-2260 | Diffuse mixed lymphoma, B lymphoblast | 0.28 |
| RL | CRL-2261 | Non-Hodgkin's lymphoma, B lymphoblast | 0.37 |
| MOLT-4 | CRL-1582 | Acute lymphoblastic leukemia (ALL) | 0.00019 |
| U-87-MG | HTB-14 | Glioblastoma, brain | 0.4 |
| HT-1080 | CCL-121 | Fibrosarcoma | 0.00025 |
| MV-4-11 | CRL-9591 | biphenotypic B myelomonocytic leukemia | 0.26 |

Pegcrisantaspase in Oncolines

| Cell line | ATCC ref. | Disease | Effect₂₀ (IU/mL) |
|---|---|---|---|
| KG-1 | CCL-246 | Acute myelogenous leukemia (AML) | 0.00018 |
| HL-60 | CCL-240 | Acute promyelocytic leukemia | 0.00028 |
| THP-1 | TIB-202 | Acute monocytic leukemia | 0.012 |
| DB | CRL-2289 | Large cell lymphoma, B lymphoblast | 0.059 |
| HT | CRL-2260 | Diffuse mixed lymphoma, B lymphoblast | 0.033 |
| RL | CRL-2261 | Non-Hodgkin's lymphoma, B lymphoblast | 0.037 |
| MOLT-4 | CRL-1582 | Acute lymphoblastic leukemia (ALL) | 0.00019 |
| U-87-MG | HTB-14 | Glioblastoma, brain | 0.057 |
| HT-1080 | CCL-121 | Fibrosarcoma | 0.013 |
| MV-4-11 | CRL-9591 | biphenotypic B myelomonocytic leukemia | 0.0088 |

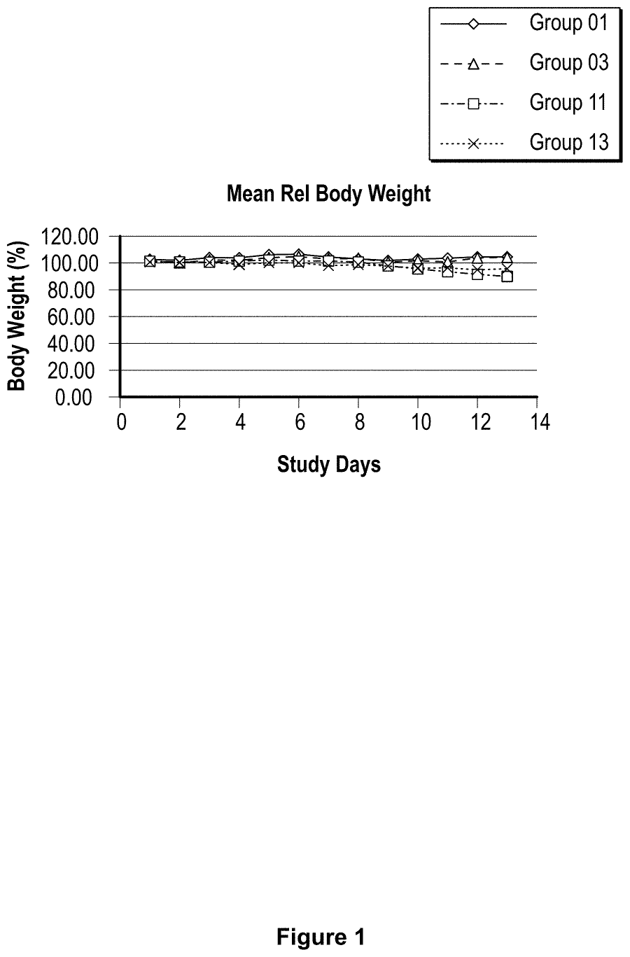

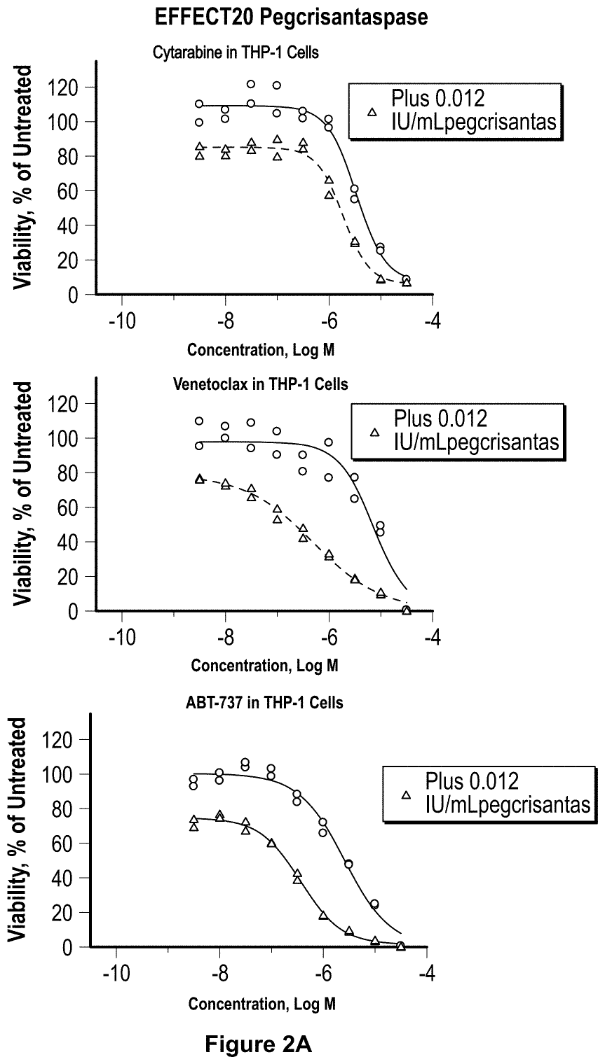

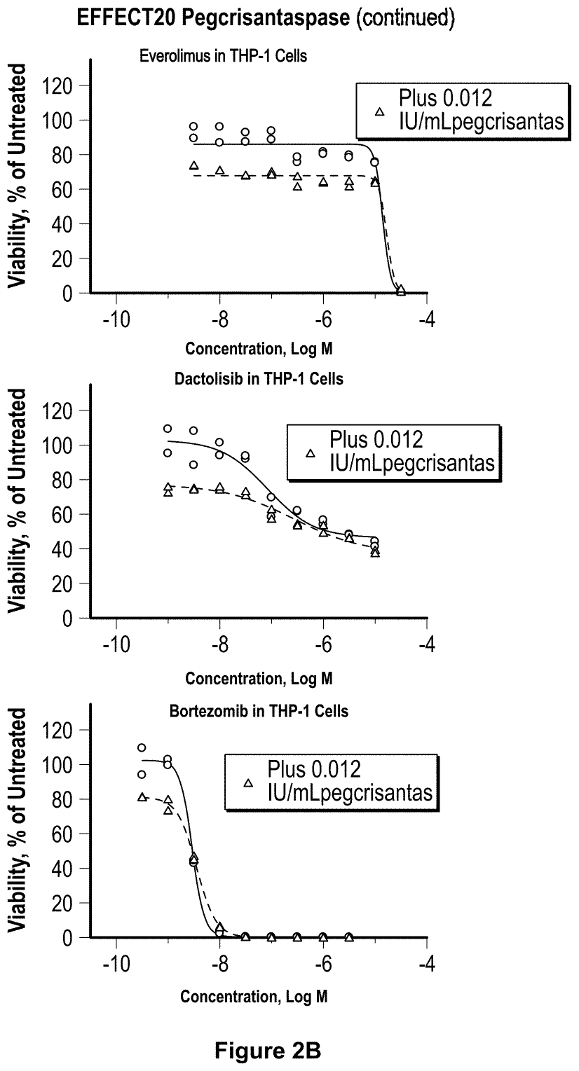

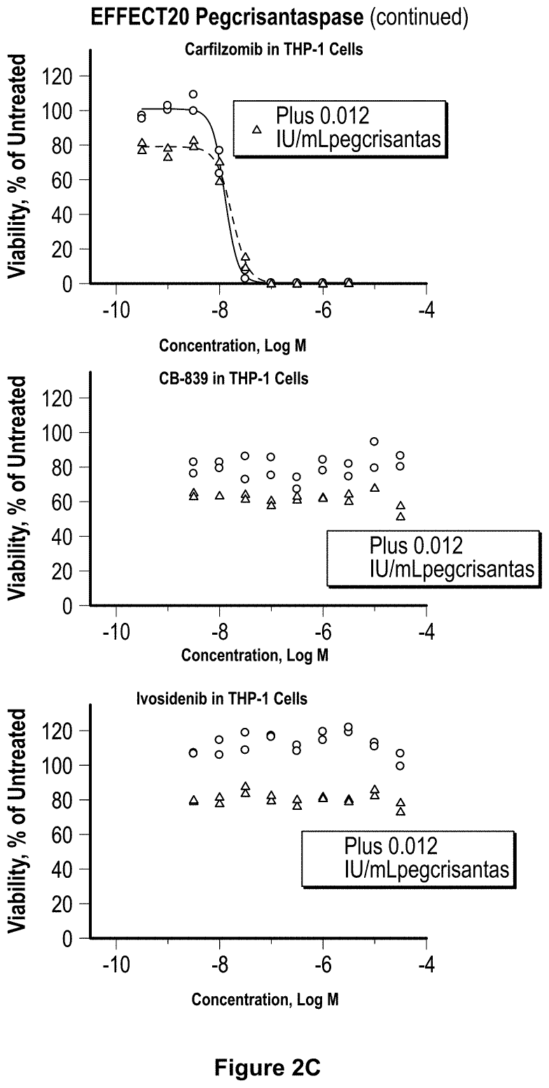

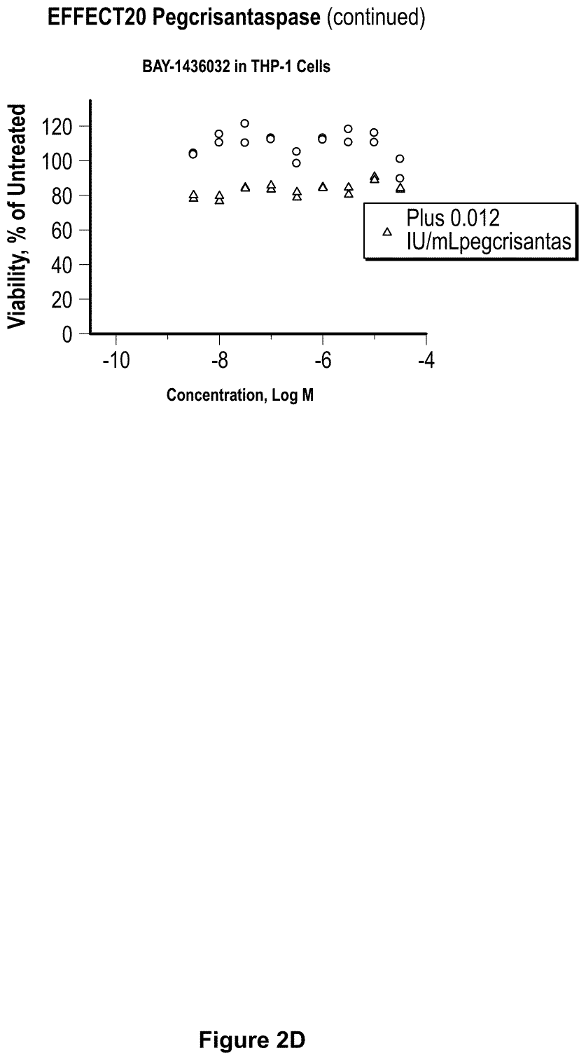

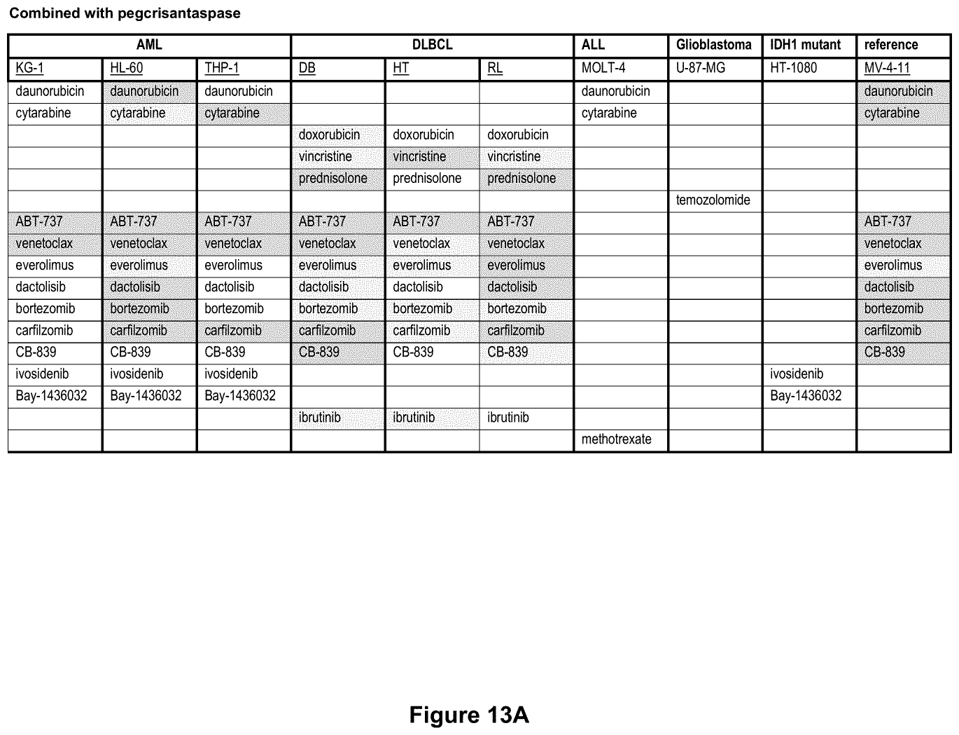

mPEG-r-crisantaspase conjugate (Pegcrisantaspase; see FIG. 13A) or Oncaspar® (see FIG. 13B) were tested with other agents that are typically used in the standard of care (SOC) for AML or DLBCL. There was an increased effect in AML when used with daunorubicin, cytarabine, ABT-737, Venetoclax, dactolisib, bortezomib, and carfilzomib. Additionally, there was an increased effect in DLBCL when used with vincristine, prednisolone, ABT-737, venetoclax, everolimus, dactolisib, bortezomib, carfilzomib, and CB-839. See the tables depicted in FIG. 13A-FIG. 13B. Grey shading indicates synergistic activity. Light grey shading indicates one experiment and dark grey indicates two experiments.
Example 3: mPEG-r-crisantaspase conjugates (Pegcrisantaspases) were tested in vivo with cytarabine and daunorubicin. Groups of 5 mice each were given mPEG-r-crisantaspase (PegC) as a single agent (5 & 50 IU/kg) and given in combination with SOC agent cytarabine (50 mg/kg once a day for 5 days followed by 2 days rest for 2 cycles) and daunorubicin (1 mg/kg administered weekly for 2 weeks). These doses were well tolerated. See FIG. 1. Group 1 is PBS control, Group 3 is PegC, Group 11 is Daunorubicin plus PegC and Group 13 is Daunorubicin. The approximate 10% decrease in mean relative body weight was due to daunorubicin.
Example 4: The present example was conducted in a manner similar to Example 1 but mPEG-r-crisantaspase conjugates (Pegcrisantaspases) were tested in combination with other compounds. FIG. 2 shows that Pegcrisantaspase potentiates the effect of cytarbine, venetoclax, and ABT-737, indicating synergy.
Example 5: mPEG-r-crisantaspase conjugates (Pegcrisantaspases) was tested in combination with ABT-737 against HL-60 cell line.

Plate preparation. The stocks of the mixtures and single agents were diluted in DMSO or 0.9% sodium chloride to generate a 7-points dose-response series. After further 31.6 times dilution in 20 mM sterile Hepes buffer pH 7.4 (reference compounds) or medium (pegcrisantaspase), 5 μl of pegcrisantaspase solution, and 5 μl of reference compound was added to 40 μl pre-plated cells in duplicate in a 384-well assay plate. The final DMSO concentration during incubation was 0.4% in all wells. Final assay concentrations range, for the single agents, between 10 and 0.01 times their IC₅₀ (10 and 0.01 IC₅₀ equivalents).

Cell proliferation assay. A cell assay stock was thawed and diluted in appropriate medium and dispensed in a 384-well plate, depending on the cell line used, at a concentration of 800-3200 cells per well in 45 μl medium: i.e., DB: 800 cells per well; RL: 1000 cells per well; MV-4-11: 1600 cells per well; KG-1, HL-60 and HT 3200 cells per well. For each used cell line the cell density was optimized previously. The margins of the plate were filled with phosphate-buffered saline. Plated cells were incubated in a humidified atmosphere of 5% CO₂ at 37° C. After 24 hours, 5 μl of pegcrisantaspase solution, and 5 μl of reference compound was added, and plates were further incubated for another 72 hours. After 72 hours, plates were cooled in 30 minutes to room temperature and 25 μl of ATPlite 1Step™ (PerkinElmer) solution was added to each well, and subsequently shaken for 2 minutes. After 5 minutes of incubation in the dark at room temperature, the luminescence was recorded on an Envision multimode reader (PerkinElmer).

Controls: t=0 signal. On a parallel plate, 40 μl cells were dispensed in quadruplicate and incubated in a humidified atmosphere of 5% CO₂ at 37° C. After 24 hours, plates were cooled to room temperature in 30 minutes. 5 μl DMSO-containing Hepes buffer, 5 μl 0.9% sodium chloride-containing medium and 25 μl ATPlite 1Step™ solution were added and subsequently mixed for 2 minutes. Luminescence was measured after 10 minute incubation (=luminescenceₜ₌₀) in the dark.

Cell growth control. The cellular doubling times of all cell lines are calculated from the t=0 hours and t=end growth signals of the untreated cells. If the doubling time is out of specification (0.5-2.0 times deviating from historic average) the assay is invalidated.

Maximum signals. On each 384-well plate, the maximum luminescence was recorded after incubation for 72 hours without compound in the presence of 0.4% DMSO. All equivalent wells (usually 14) were averaged. This average is defined as: luminescence_{untreated,t=72h}. Dose response curves. Accurate single agent IC₅₀s are needed for combination analysis. For each single agent its dose-response signal was fitted by a 4-parameter logistics curve using XL-fit 5 (IDBS software):

luminescence=bottom+(top-bottom)/(1+10^{(logIC50-log[cpd])-hill)})

[cpd] is the compound concentration tested. hill is the Hill-coefficient. Bottom and top are the asymptotic minimum and maximum of the curve.
Combination Index (CI) determination. The CI is one of the most widely used quantitative indications of synergy. The CI evaluates the concentrations needed to achieve a fixed-effect. A CI of below 1 indicates synergy. A CI of less than 0.3 indicates strong synergy. For example, a CI of 0.1 indicates that the combination needs a ten-fold lower concentration than expected from the single agent data, to achieve the same effect level. For instance, when a potent and less potent compound with a CI of 0.1 are combined, the effective concentration of the potent compound is improved tenfold by the less potent compound.

CI is defined for a certain percentage cell viability (V), which is the signal related to a non-exposed control: V=100%×luminescence_{treated,t=72h}/luminescence_{untreated,t=72h}. The concentrations of the two compounds cpd1 and cpd2 needed to reach a certain percentage cell viability V in combination are then compared to the concentrations needed as single agents:

CI_{(100-V)}=[cpd1]ᵥ/IC_{(100-V),cpd1}+[cpd2]ᵥ/IC_{(100-V),cpd2}

For example, [cpd1]₅₀ signifies the concentration of cpd1 in a mixture that gives 50% viability. IC_{50,cpd1} would signify the IC₅₀ of cpd1 alone. The CI is labelled by %-effect, to follow conventions, so CI₇₅ signifies the CI at 25% viability
Curve shift analysis. This analysis provides a visual confirmation of synergy¹. The concentrations of the mixtures of compounds 1 and 2 (cpd1 and cpd2), and the single agents, were expressed in terms of IC₅₀ equivalents (in ‘units’ of IC₅₀):

[mix]=[cpd1]/IC_{50,cpd1}+[cpd2]/IC_{50,cpd2}

The dose-response signal was fitted by a 4-parameter logistics curve using XL-fit 5 (IDBS software)

luminescence=bottom+(top-bottom)/(1+10^{(logX-log[mix])-hill)})

Here hill is the Hill-coefficient and X the inflection point of the curve. Bottom and top are the asymptotic minimum and maximum of the curve. Because [mix] is expressed in terms of IC₅₀ equivalents, the curves of the single agents will overlap and their inflection point will lie at a value of 1. The IC₅₀ values that are used in the calculations, are those determined in parallel for the single agents.

In mixtures where synergy is absent, curves will overlap those of the single agents. In mixtures where there is synergy, curves will shift leftward towards lower IC₅₀ equivalents: the mixture appears more potent than expected on basis of the individual constituents. This is a good indicator of synergy.

Isobolograms. An isobologram is a dose-oriented plot which reveals whether drug combinations are synergistic. It is defined at a certain effect level, which is usually 75%. If the single agent curves do not achieve this efficacy level, the isobologram level is set at 50%, 30%, 25% or 20%. If single agents do not reach the 20% effect, no isobologram is drawn. On the axis, the calculated doses of the single compounds are plotted that give the pre-set growth effect. Both points are connected with a straight line (additivity line). For the drug combinations, it is calculated which dilutions give the pre-set growth effect and the concentrations of the individual components at this point are plotted in the isobologram. In case of an additive drug effect, the drug combination will lie close to the additivity line. In case of synergy or antagonism, the points will lie under or above the line, respectively.

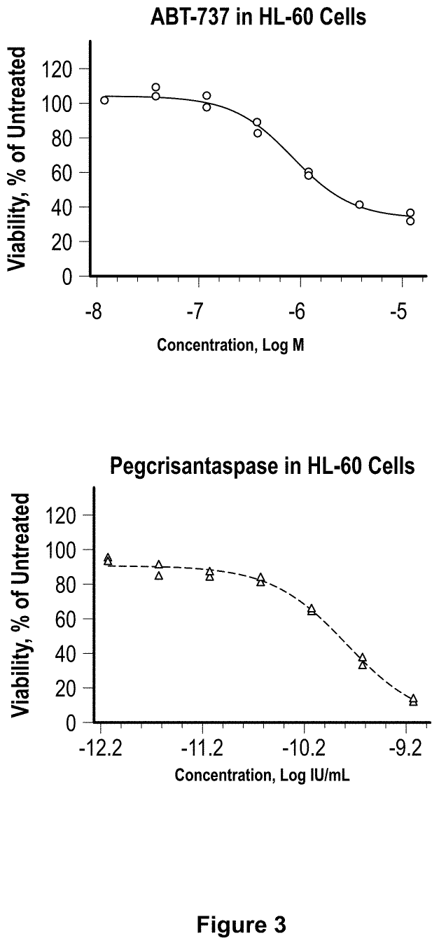

Experiments with inactive agents. In certain agreed cases, synergy experiments are performed in the presence of ‘inactive’ agents, which are compounds that do not give a dose-response curve as single agents, at the concentrations tested. The experiments are executed as described above except that the ‘inactive’ agent is added in a fixed concentration to each well of the experiment. Because the single ‘inactive’ agent shows no effect, its contribution to CI is insignificant. CI values are then based on the response of the active agents. Curve shift of the mixture is determined compared to the other, active agent. No isobologram is calculated. The dose-response curves with single agents is depicted in FIG. 3. ABT-737 has an IC50 of 835 nM and a maximal effect at 67% while pegcrisantaspase had an IC₅₀ of 0.15 nM and a maximal effect at 88%.

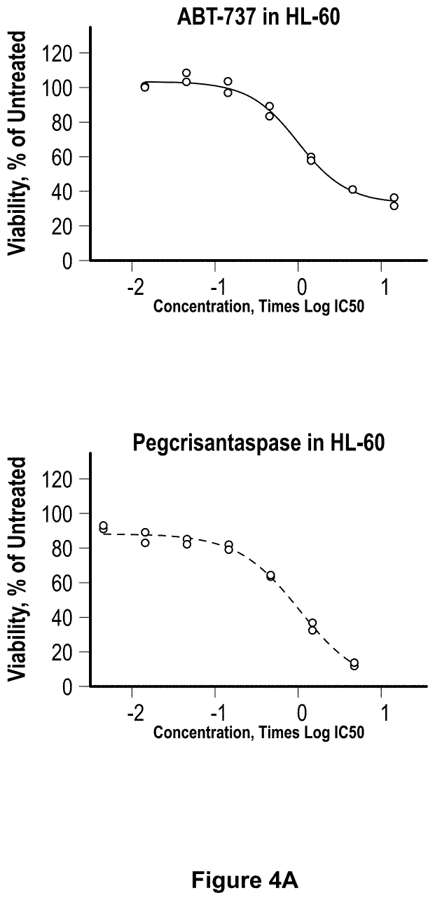

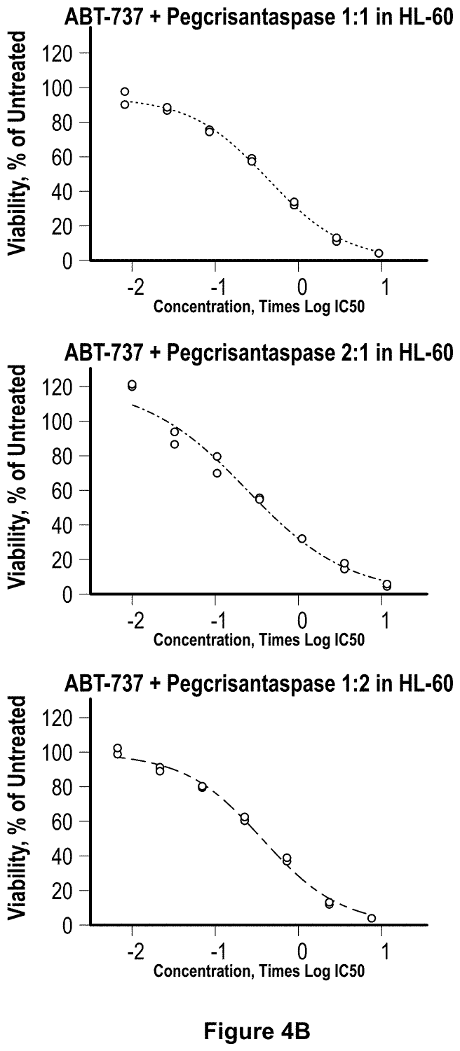

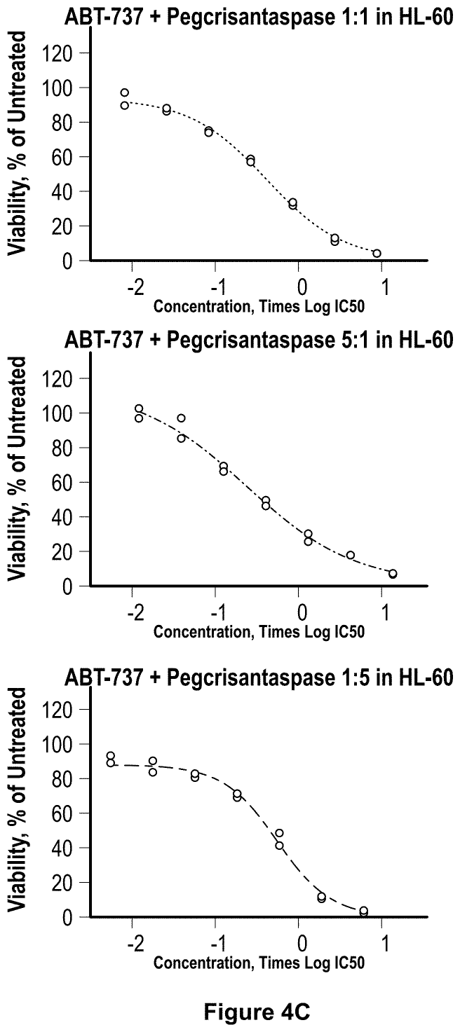

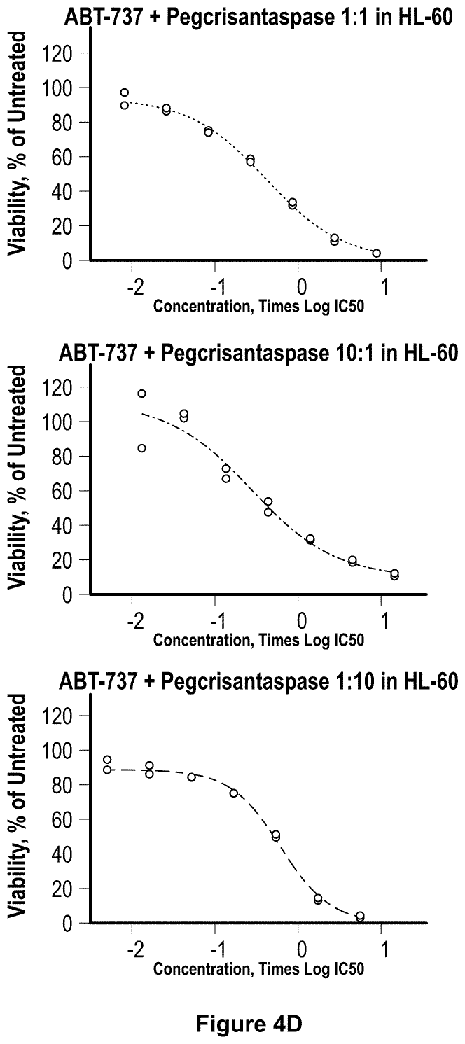

Curve shift analysis: The x-axes of the single agent curves (grey and dark grey) and the mixture curves (red, orange and pink) were translated to an IC₅₀ equivalent, based on the IC₅₀s of the single agents, and are compared to the dose-response curves of the mixture as shown in FIG. 4.

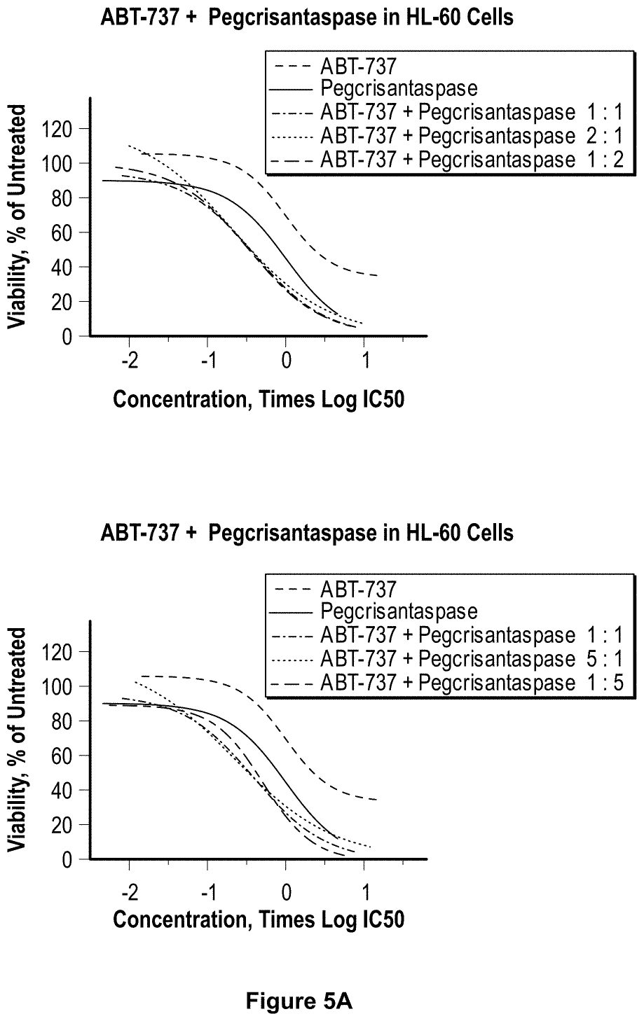

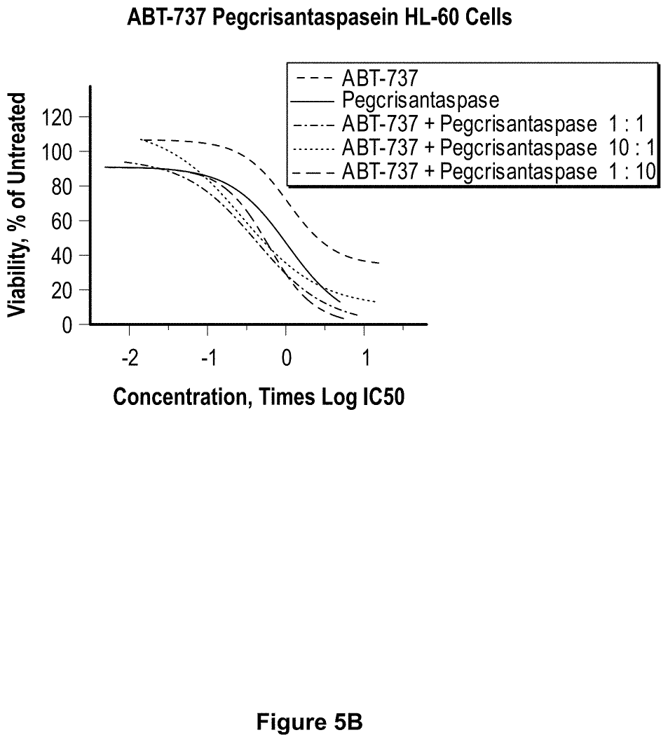

For dose-response curves on the mixtures on an IC₅₀ basis, all curves were superimposed and shifts recorded. A leftward shit of the mixtures curves compared to the single agent curves (grey and dark grey) indicates synergy, a rightward shift indicates antagonism (see FIG. 5 and tables below).

### IC₅₀ Shifts of Mixtures Compared To Single Agents

Results using the combination of pegcrisantaspase and ABT-737 are shown below. CI values calculated from the mixture data, ED₇₅ corresponds to 25% viability. A representative value is the average CI value for the three mixtures at 50% viability, which is indicated in the summary.

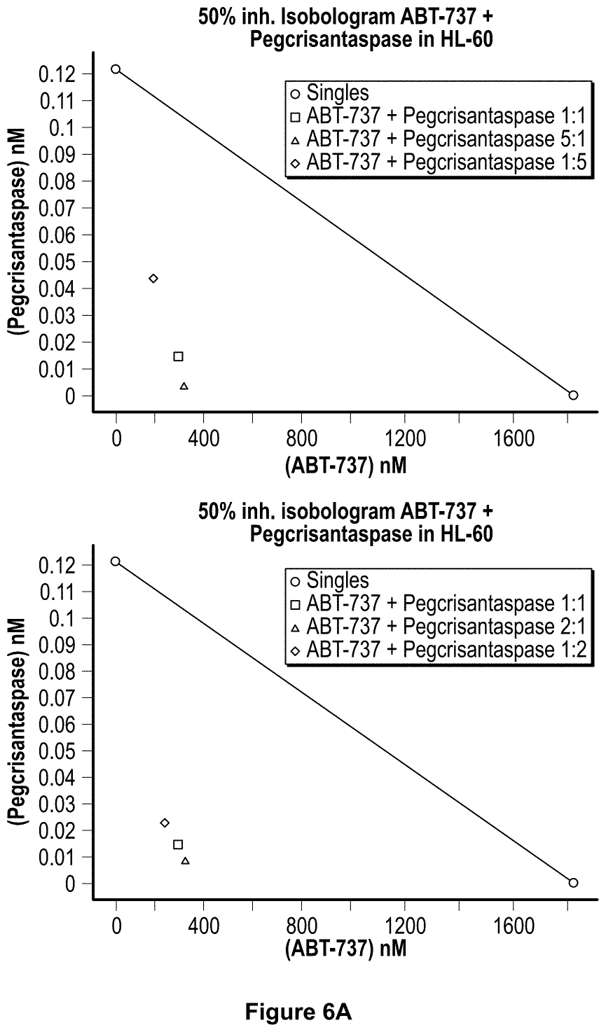

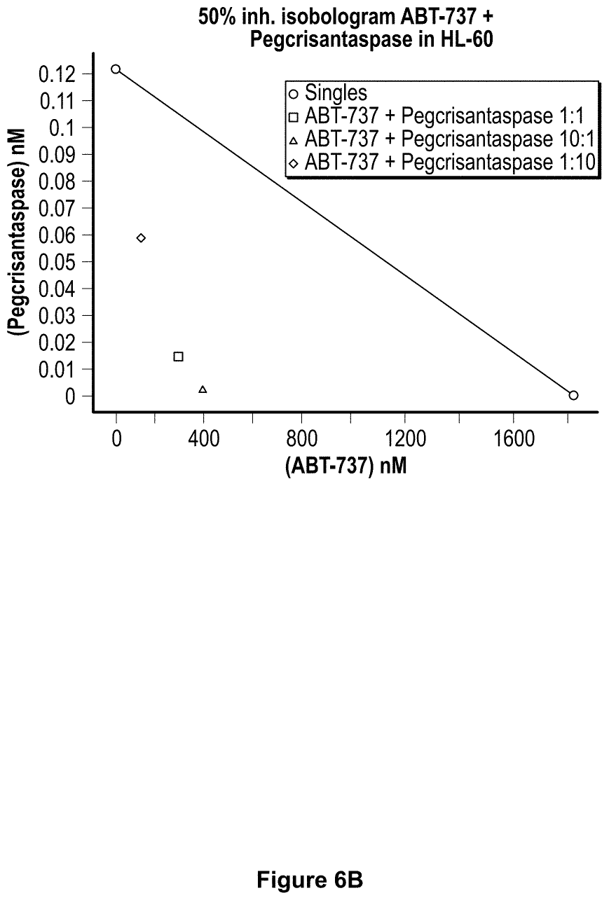

The combination data were used to generated isobolograms as shown in FIG. 6. An isobologram is a dose-oriented plot that reveals whether drug combinations are synergistic. In case of synergy, combination points lie under the straight additivity line. The concentration of pegcrisantaspase is shown in IU/mL. The additivity line (dark grey) indicates concentration combinations that would give theoretical additivity. Drug combinations are plotted as the red, pink and orange dots. In summary, strong synergy between pegcrisantaspase and ABT-737 in HL-60 cell line was found as shown below.

| | IC₅₀ | | IC₅₀ | | Average CI value | Average CI value | Average CI value | Curve |
|---|---|---|---|---|---|---|---|---|
| Compound | (IU/mL) | Therapeutic | (nM) | Cell line | 1:1, 1:2 and 2:1* | 1:1, 1:5 and 5:1* | 1:1, 1:10 and 10:1* | shift |
| pegcrisantaspase | 0.070 | ABT-747 | 1213 | HL-60 | 0.26 | 0.29 | 0.33 | yes |

*Average Combination Index of the mixtures at ED50; CI = 1.0: no synergy; CI < 1.0: synergy; CI < 0.3: strong synergy; CI > 1.5: antagonistic
ND: not determined, tested compound had an efficacy <20%
NA: not applicable

Example 6: The present example was conducted in a manner similar to Example 5 but the synergy with additional anti-cancer agents in different cell types were tested as shown below.

| AML | | | DLBCL | | |
|---|---|---|---|---|---|
| Cell line | anti-cancer agent | conclusion | Cell line | anti-cancer agent | conclusion |
| KG-1 | daunorubicin | synergy | DB | ABT-737 | synergy |
| KG-1 | cytarabine | synergy | DB | venetoclax | synergy |
| KG-1 | ABT-737 | strong synergy | DB | carfilzomib | synergy |
| KG-1 | venetoclax | strong synergy | DB | prednisolone | no synergy |
| KG-1 | dactolisib | strong synergy | DB | vincristine | strong synergy |
| KG-1 | bortezomib | synergy | HT | carfilzomib | no synergy/antagonism |
| KG-1 | carfilzomib | synergy | HT | vincristine | synergy |
| HL-60 | daunorubicin | antagonism | HT | ABT-737 | synergy |
| HL-60 | cytarabine | synergy | HT | venetoclax | no synergy |
| HL-60 | ABT-737 | strong synergy | RL | ABT-737 | synergy |
| HL-60 | venetoclax | strong synergy | RL | venetoclax | synergy |
| HL-60 | dactolisib | synergy | RL | carfilzomib | synergy |
| HL-60 | bortezomib | antagonism | RL | prednisolone | synergy |
| HL-60 | carfilzomib | antagonism | RL | vincristine | synergy |
| MV-4-11 | daunorubicin | | | | |
| MV-4-11 | cytarabine | | | | |
| MV-4-11 | ABT-737 | | | | |
| MV-4-11 | venetoclax | | | | |
| MV-4-11 | dactolisib | | | | |
| MV-4-11 | bortezomib | | | | |
| MV-4-11 | carfilzomib | | | | |

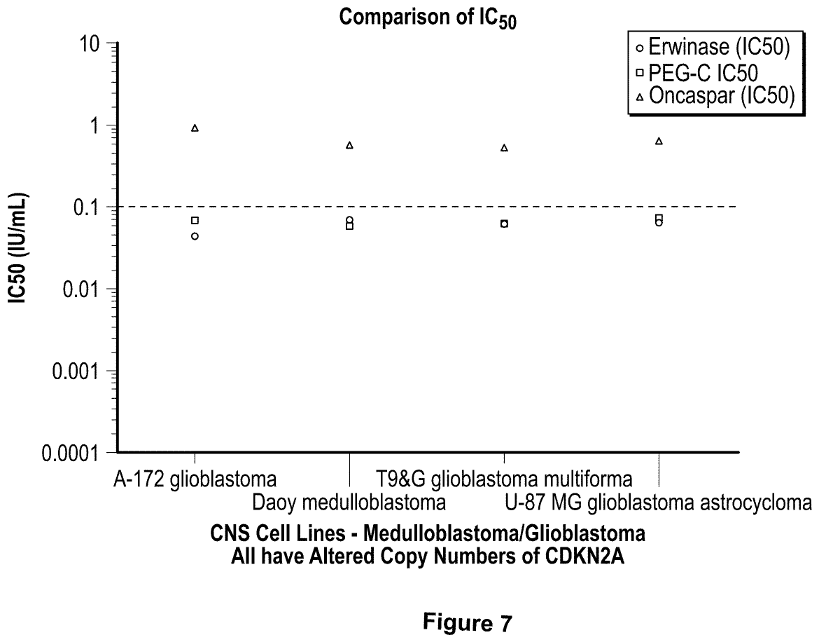

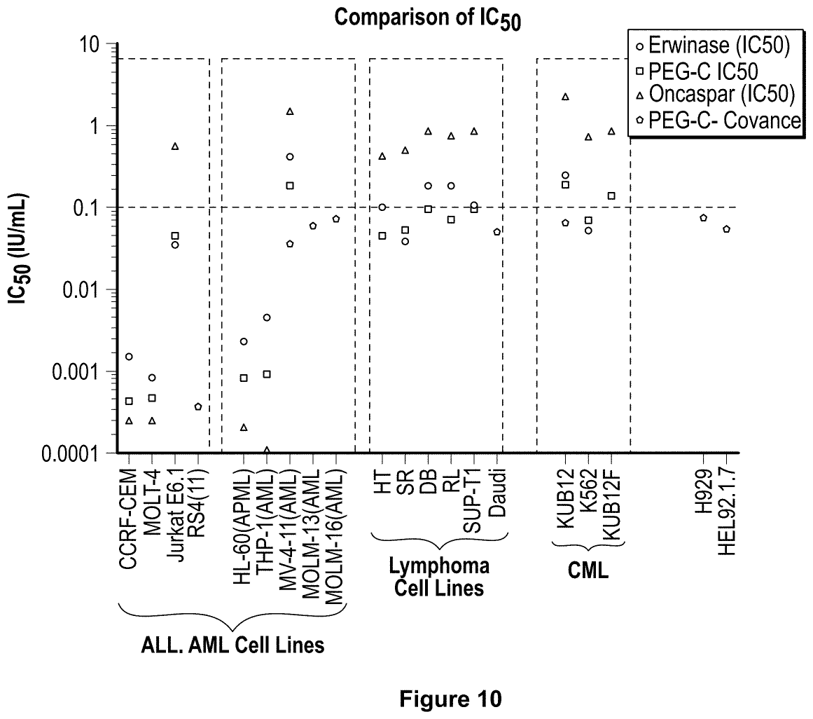

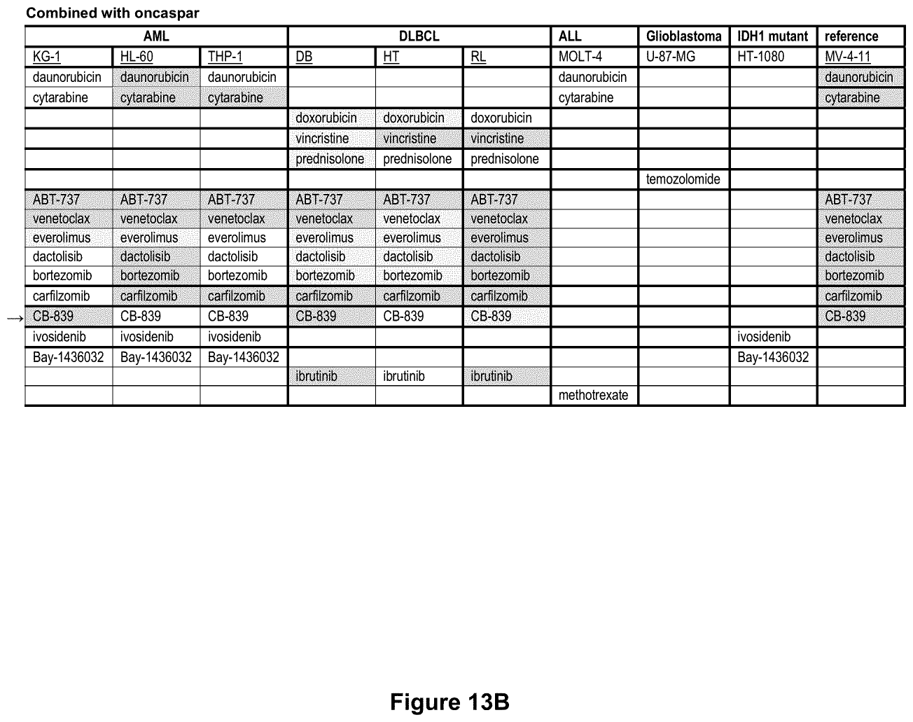

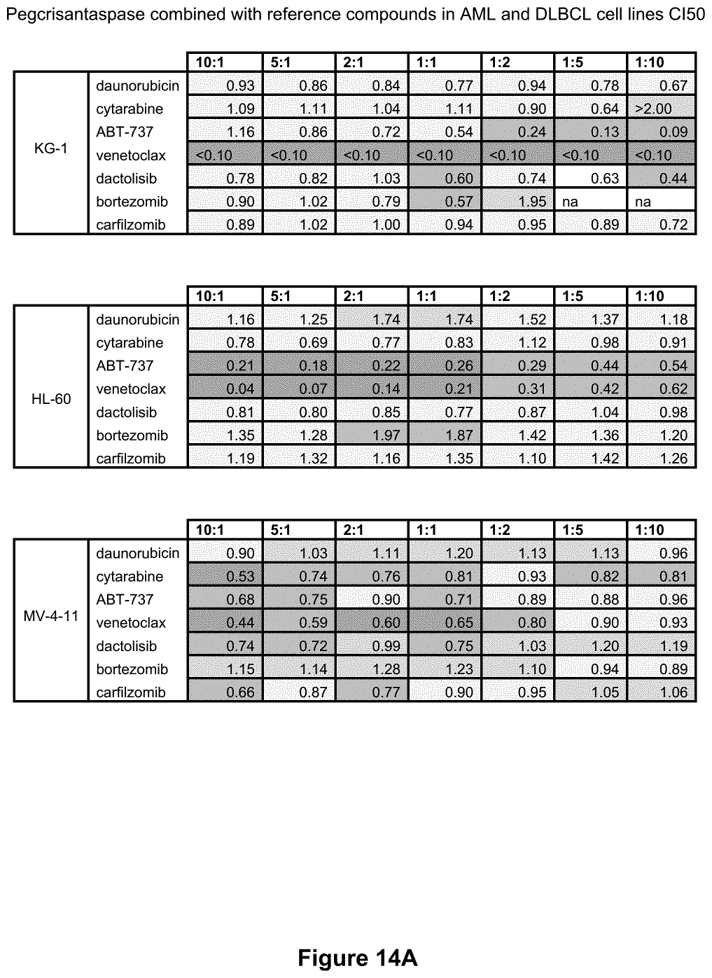

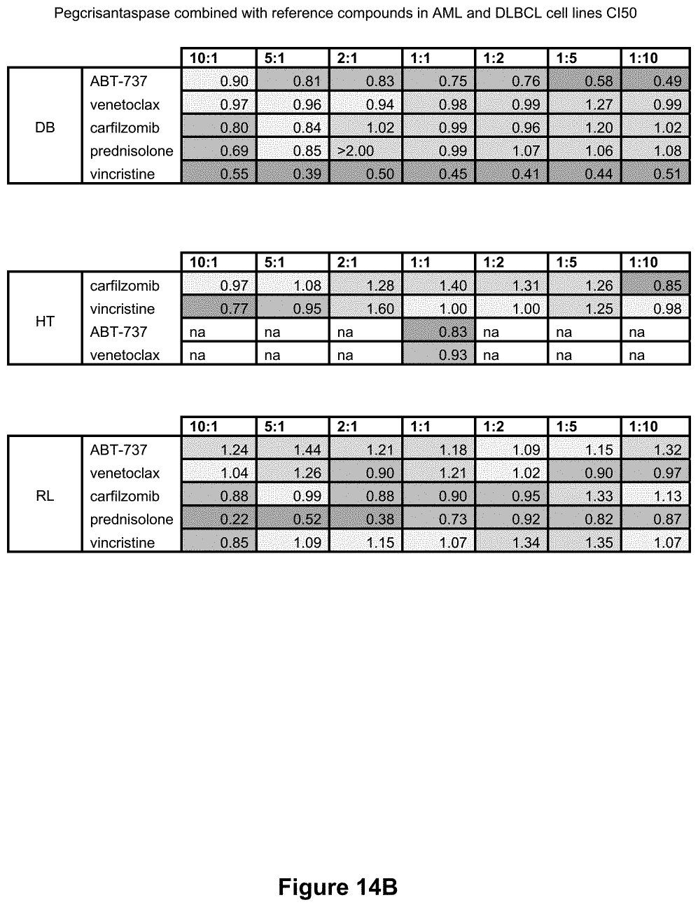

Example 7: The present example was conducted in a manner similar to Example 1 but mPEG-r-crisantaspase conjugates were tested for activity against CNS cell lines, including for example, glioblastoma, medulloblastoma, glioblastoma multiforma and glioblastoma astrocytoma. Results are displayed in FIG. 7. Additional experiments using different cell lines were performed, and the results are displayed in FIG. 10.
Example 8: mPEG-r-crisantaspase conjugates (Pegcrisantaspases) in combination of additional compounds were tested against AML (acute myeloid leukemia) and DLBCL (diffuse large B-cell lymphoma) cell lines in accordance with the methods described in Example 1. Results are shown in FIG. 14A-FIG. 14B. KG-1, HL-60 and MV4-11 are AML cell lines, and DB, HT and RL are DLBCL cell lines. The combination data with pegcrisantaspase and venetoclax showed strong synergy in the AML cell lines.

### Example 9

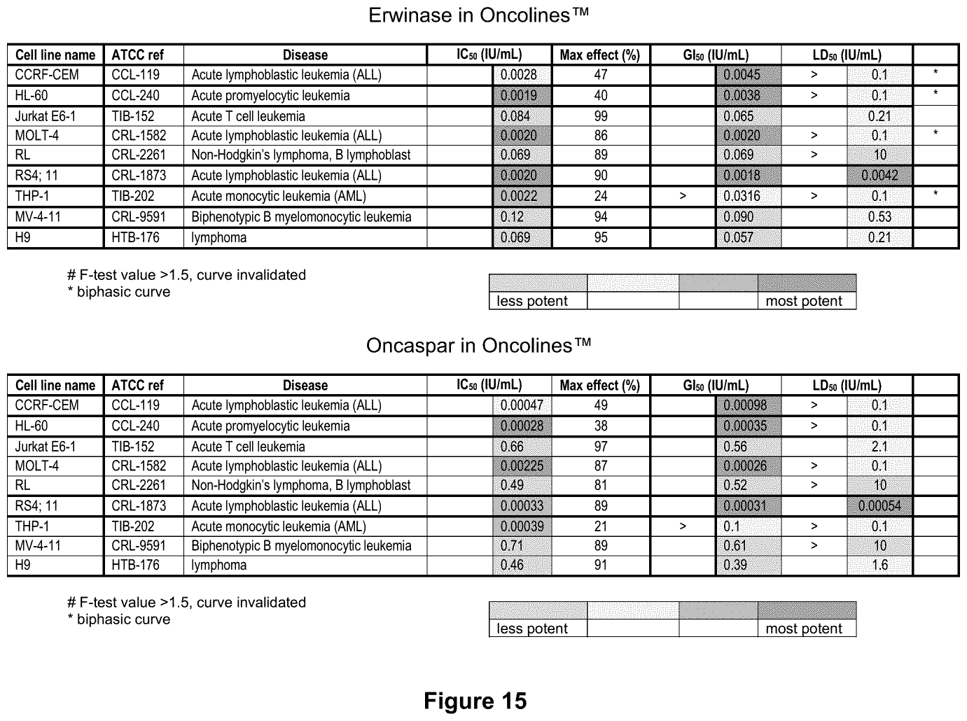

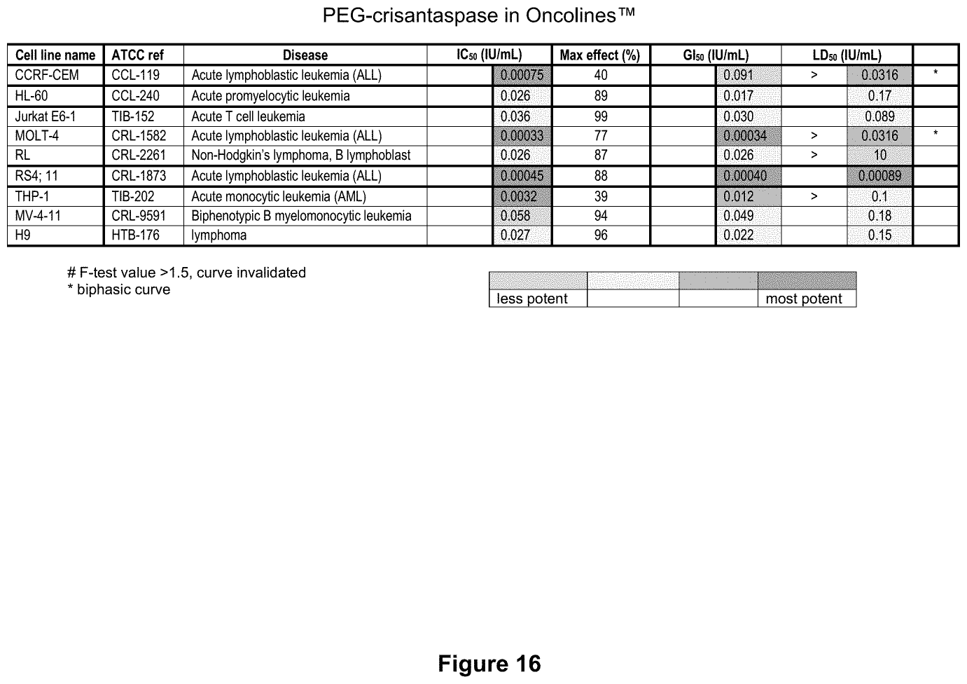

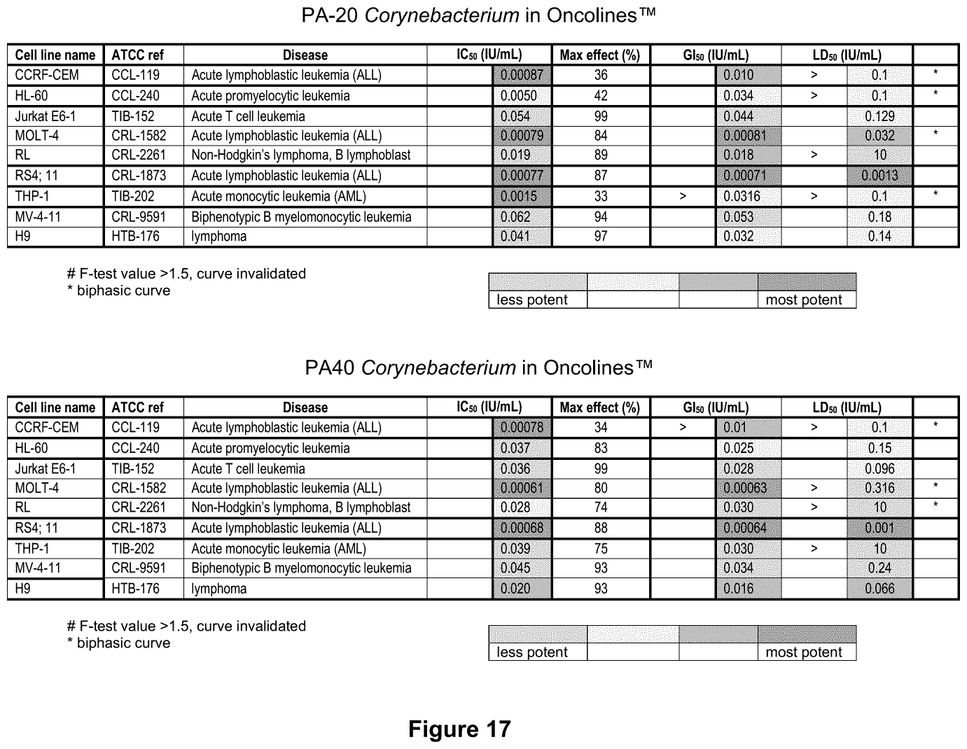

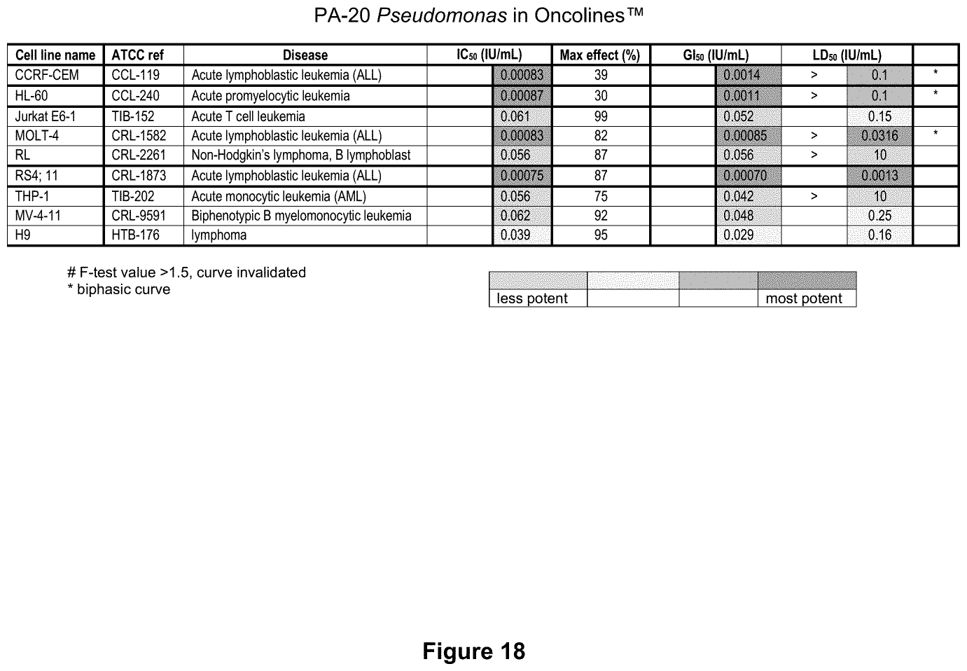

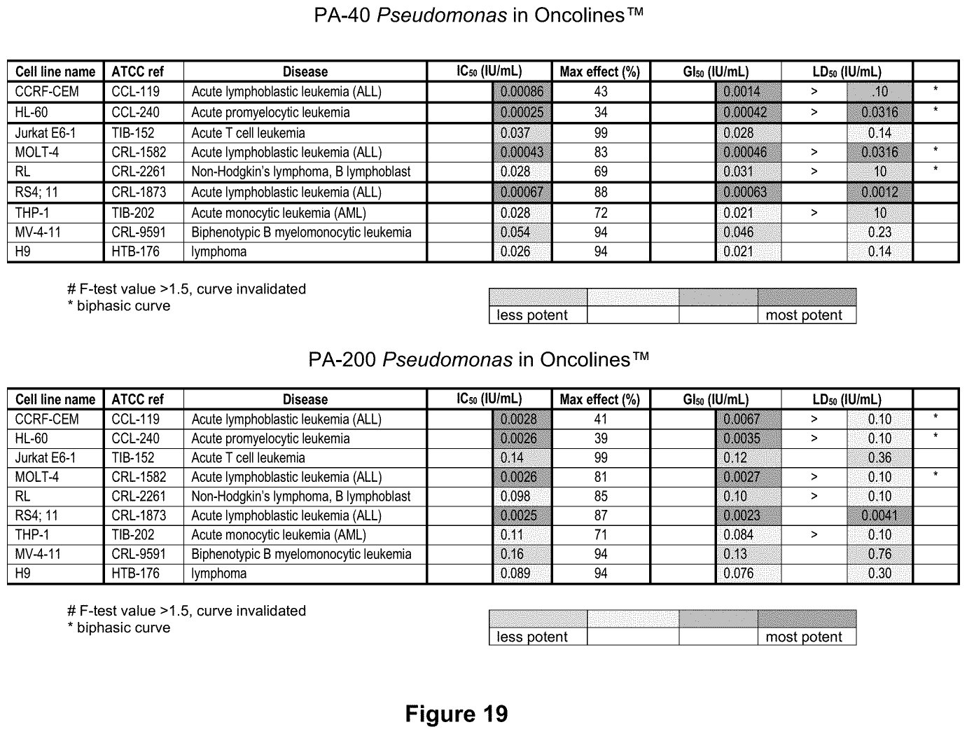

Pasylated conjugates of crisantaspases were tested against pegylated (PEG-crisantaspase) and non-pegylated (Erwinase) versions of crisantaspases along with E. coli L-asparaginase (Oncaspar) in multiple cell lines in accordance with the methods described in Example 1. PA-20 and PA-40 are pasylated crisantaspase conjugates produced in Corynebacterium or Pseudomonas expression systems and PA-200 is a pasylated fusion protein produced in Pseudomonas expression system. The PA-20, PA-40, PA-200 and PA-400 constructs are SEQ ID NO: 2, 3, 6 and 7. Results are shown in FIG. 15-FIG. 19. CCRF-CEM, MOLT-4 and RS4:11 are all AML cell lines, Jurkat E6-1 is an acute T-cell leukemia cell line, HL-60 is an acute promyelocytic leukemia cell line, MV4-11 is a biphenotypic B-cell myelomonocytic leukemia cell line, THP-1 is an AML cell line, RL is a non-Hodgin's lymphoma cell line, and H9 is a lymphoma cell line.
Those skilled in the art will recognize, or be able to ascertain using no more than routine experimentation, many equivalents to the specific embodiments of the invention described herein. Such equivalents are intended to be encompassed by the following claims.

1. A method of treating a disease in a patient comprising administering to the patient an effective amount of a conjugate comprising:
(i) a protein with at least 95% sequence identity to SEQ ID NO:1, linked to a (ii) polyethylene glycol (PEG) molecule, with a molecular weight of 500 to 9000 Da,
wherein the disease is colon cancer.

2. The method of claim wherein the protein has at least about 96%, 97%, 98%, 99%, or 100% sequence identity to SEQ ID NO: 1.

3. The method of claim wherein the protein has 100% sequence identity to SEQ ID NO: 1.

4. The method of claim wherein the PEG molecule has a molecular weight of about 5000 Da, 4000 Da, 3000 Da, 2500 Da, or 2000 Da.

5. The method of claim wherein the conjugate has an in vitro activity of at least 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to protein when not conjugated to the PEG molecule.

6. The method of claim 1, wherein the conjugate has an L-asparagine depletion activity at least about 10, 20, 30, 40, 50, 60, 70, 80, 90, or 100 times more potent than the protein when not conjugated to the PEG molecule.

7. The method of claim wherein the conjugate depletes plasma L-asparagine levels to an undetectable level for at least about 12, 24, 48, 96, 108, or 120 hours.

8. The method of claim wherein the conjugate has a longer in vivo circulating half-life compared to protein when not conjugated to the PEG molecule.

9. The method of claim 1, wherein the conjugate has a longer t½ than pegaspargase administered at an equivalent protein dose.

10. The method of claim wherein the conjugate has a t½ of at least about 58 to 65 hours at a dose of about 50 μg/kg on a protein content basis, and a t½ of at least about 34 to about 40 hours at a dose of about 10 μg/kg on a protein content basis, following iv administration in mice.

11. The method of claim wherein the conjugate has a t½ of at least about 100 to about 200 hours at a dose ranging from about 10,000 to about 15,000 IU/m² (about 20-30 mg protein/m²).

12. The method of claim 1, wherein the conjugate has a greater area under the curve (AUC) compared to the protein when not conjugated to the PEG molecule.

13. The method of claim 1, wherein the conjugate has a mean area-under-curve (AUC) that is at least about 3 times greater than pegaspargase at an equivalent protein dose.

14. The method of claim wherein the PEG molecule is covalently linked to one or more amino groups of the protein.

15. The method of claim 14, wherein the PEG molecule is covalently linked to the one or more amino groups by an amide bond.

16. The method of claim wherein the PEG molecule is covalently linked to at least from about 40% to 100% of accessible amino groups of the protein or at least from about 40% to 90% of total amino groups of the protein.

17. The method of claim 1, wherein the conjugate has the formula:
Asp-[NH—CO—(CH₂)ₓ—CO—NH-PEG]ₙ
wherein Asp is the protein, NH is one or more amino groups of lysine residues and/or N-termini of the Asp, PEG is the PEG molecule, n is a number that represents at least about 40% to about 100% of accessible amino groups in the Asp, and x is an integer ranging from about 1 to 8.

18. The method of claim wherein the PEG molecule is monomethoxy-polyethylene glycol (mPEG).

19. The method of claim wherein disease is a cancer the colon cancer is colon adenocarcinoma.

20. (canceled)

21. The method of claim wherein the colon cancer is colon carcinoma, colon adenocarcinoma, or colorectal adenocarcinoma.

22. The method of claim 1, wherein the conjugate is administered at an amount of about 5 U/kg body weight to 50 U/kg body weight.

23. The method of claim 1, wherein the conjugate is administered at a dose ranging from about 10,000 to 15,000 IU/m².

24. The method of claim 1, wherein the administration is intravenous or intramuscular and is once per week, twice per week, or three times per week.

25. The method of claim 1, wherein the conjugate is administered as monotherapy.

26. The method of claim 23, wherein the conjugate is administered as part of a combination therapy.

27. The method of claim 25, wherein the conjugate is administered as part of a combination therapy with Oncaspar®, daunorubicin, cytarabine, Vyxeos®, ABT-737, Venetoclax, dactolisib, bortezomib, carfilzomib, vincristine, prednisolone, everolimus, and/or CB-839.

28. The method of claim 1, wherein the patient receiving treatment has had a previous hypersensitivity to an Escherichia coli asparaginase or a PEGylated form thereof or to an Erwinia asparaginase.

29. The method of claim 1, wherein the patient receiving treatment has had a disease relapse, in particular a relapse that occurs after treatment with an Escherichia coli asparaginase or a PEGylated form thereof.

30. The method of claim 1, wherein the colon cancer includes a mutation in a gene selected from NRAS, PTEN, ARHGAP35, MLH1, CHD4, CDKN2A, COND1, ASXL1, ERBB2, KEAP1, an ACVR1B, SMARCA4, FBXW7, MSH6, PCU2F2, PPMID, NSD1, TNFAIP3, ARID1A, CTCF, JAK1, MLL4, EP300, STIK11, SPEN, CTNNB1, SMC3, KRAS, LRP1B or a combination thereof.
